SEQUENCE LISTING (16 sequences)

SEQ ID NO: 1 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 2 - protein, 20 aa, Homo sapiens
KDAPDHQELNLDVSLQLPSR

SEQ ID NO: 3 - protein, 20 aa, Homo sapiens
SEETKENEGFTVTAEGKGQG

SEQ ID NO: 4 - protein, 565 aa, Homo sapiens
ARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 5 - protein, 343 aa, Homo sapiens
SEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 6 - protein, 3 aa, Artificial Sequence
  Artificial tripeptide linker for attaching a carrier to an immunogen.
GGC

SEQ ID NO: 7 - protein, 20 aa, Mus musculus
TDVPDHKDLNMDVSFHLPSR

SEQ ID NO: 8 - protein, 20 aa, Mus musculus
SEETKQNEAFSLTAKGKGRG

SEQ ID NO: 9 - protein, 23 aa, Homo sapiens
NLDVSLQLPSRSSKITHRIHWES

SEQ ID NO: 10 - protein, 16 aa, Homo sapiens
WESASLLRSEETKENE

SEQ ID NO: 11 - protein, 4 aa, Homo sapiens
RRRR

SEQ ID NO: 12 - protein, 5 aa, Homo sapiens
QLPSR

SEQ ID NO: 13 - protein, 5 aa, Homo sapiens
SEETK

SEQ ID NO: 14 - protein, 8 aa, Artificial Sequence
  Amino acids 1299-1303 of  C3 Precursor sequence (SEQ ID NO1) linked to an artificial CGG linker.
CGGQLPSR

SEQ ID NO: 15 - protein, 8 aa, Artificial Sequence
  Amino acids 1321-1325 of  C3 Precursor sequence (SEQ ID NO1) linked to an artificial GGC linker.
SEETKGGC

SEQ ID NO: 16 - protein, 8 aa, Artificial Sequence
  Amino acids 1321-1325 of  C3 Precursor sequence (SEQ ID NO1) linked to an artificial GGC linker with a C-terminal hydroxyl (-OH) group.
SEETKGGC

(19) United States
(12) Patent Application Publication
(10) Pub. No.: US 2014/0186371 A1
(43) Pub. Date: Jul. 3, 2014

(54) COMPOSITIONS AND METHODS FOR TREATING DISEASES OF PROTEIN AGGREGATION INVOLVING IC3B DEPOSITION

(71) Applicants: Guriqbal S. Basi, Palo Alto, CA (US); Robin Barbour, Walnut Creek, CA (US)

(72) Inventors: Guriqbal S. Basi, Palo Alto, CA (US); Robin Barbour, Walnut Creek, CA (US)

(21) Appl. No.: 14/110,137

(22) PCT Filed: Apr. 6, 2012
(86) PCT No.: PCT/US12/32635

§ 371 Date: Feb. 5, 2014

Related U.S. Application Data
(60) Provisional application No. 61/473,107, filed on Apr. 7, 2011.

(51) Int. Cl. C07K 16/18; G01N 33/543; A61K 39/00

(52) U.S. Cl. CPC C07K 16/18; A61K 39/0005; G01N 33/543
USPC 424/172.1; 530/387.3; 530/389.1; 424/184.1; 530/387.9; 424/185.1; 435/7.92

(57) ABSTRACT

The invention provides antibodies that preferentially bind to iC3b relative to C3b. These antibodies serve to reduce the toxicity of this fragment and find use in treatment and prophylaxis of a variety of diseases associated with deposits of the fragment.

### CROSS-REFERENCE TO RELATED APPLICATION

This application claims priority to U.S. Provisional Patent Application No. 61/473,107, filed Apr. 7, 2011, which is incorporated by reference in its entirety for all purposes.

### REFERENCE TO A SEQUENCE LISTING

The Sequence Listing written in file Sequence_Listing_for_{—}057450-417337.txt is 24,425 bytes and was created on Apr. 6, 2012. The information contained in this file is hereby incorporated by reference.

### BACKGROUND OF THE INVENTION

Complement is an auxiliary system in immunity and antimicrobial defense including more than 35 plasma or membrane proteins. Complement is predominantly activated by a cascade of proteolytic steps. The three complement activation pathways (classical, lectin and alternative) all lead to the activation of complement protein C3, which is cleaved into fragments C3a and C3b.

The classical complement activation pathway begins with antibodies bound to a pathogen surface, which in turn bind the C1q complement component. This sets off a serine protease catalytic cascade involving serum complement proteins that ultimately cleaves C3 to its active form, C3b. The lectin pathway is activated via recognition of carbohydrate motifs by lectin proteins. The alternative pathway activates complement by direct reaction of an internal C3 ester with recognition motifs on the pathogen surface.

C3 is a heterodimer of an alpha chain and a beta chain held together by a disulfide bond formed between the N-terminal regions of the two chains. Another disulfide bond exists between the N-terminal region and the C-terminal region of the alpha chain. The alpha chain contains a thioester between a cysteine and a glutamine residue that are three positions apart. This thioester allows for the activation-dependent formation of covalent bonds. Activation of C3 by C3-convertases yields C3a and C3b. C3b changes its conformation to expose the internal thioester bond and binds to nearby nucleophils (acceptor molecules). This is the initial step in complement-mediated opsonization, that is, the rendering of pathogens subject to phagocytosis by, for example, macrophages. C3b has the ability to self-amplify and circulating levels in plasma are subject to tight control. Cleavage and consequent inactivation of C3b is achieved by factor I and a cofactor, yielding iC3b, which is normally then further degraded by factor I and CR1. Comparison of C3b to C3 demonstrates that the molecule undergoes major conformational rearrangements with each proteolysis, which exposes not only the internal thioester bond, but additional new surfaces of the molecule that can interact with cellular receptors.

To prevent unwanted complement activation in the body, most mammalian cells are equipped with regulators that block complement amplification on host cells. Without these intrinsic regulators, the generation of activated complement proteins facilitates inflammation and tissue damage. Thus, non-cellular surfaces that lack intrinsic complement regulators are especially prone to complement attack and are fully dependent on protection by soluble complement regulators in serum. Unregulated complement activation has been associated with various chronic inflammatory diseases and degenerative diseases. The complement split products C3a and C5a, which function as a chemo-attractant and activators of neutrophils and inflammatory macrophages via the C3a and C5a receptors, are dominant in this inflammatory cascade. Complement activation has been shown to be an important component capable of driving chronic inflammation in immune-complex mediated diseases such as membrane-proliferative glomerulonephritis, nephrotoxic nephritis, and arthritis.

AMD is the primary cause of blindness in the elderly, affecting 30-50 million elderly individuals worldwide. Genetic association studies link polymorphisms in factor H, factor B, and C3 with AMD. The lack of regulation of the alternate pathway for complement activation is postulated as a major cause underlying the two primary clinical forms of AMD: wet (exudative) and dry. Wet AMD is the less common form (10-20% of total AMD cases) and is characterized by choroidal neo-vascularization of retinal pigment epithelial cell layer in the retina.

AMD is a disorder characterized by extracellular lipoproteinaceous deposits known as drusen. Drusen forms in eye tissue between the basal surface of retinal pigment epithelial cells and a basement membrane complex called Bruch's membrane and includes lipofusin pigments from degenerating RPE cells and plasma components. Among the constituents of drusen are complement proteins depositing on the lipofuscin, which originates from degenerating retinal pigment epithelial cells, and Aβ peptide (Johnson, Leitner et al. 2002; Dentchev, Milam et al. 2003; Yoshida, Ohno-Matsui et al. 2005; Luibl, Isas et al. 2006). Drusen is immunoreactive for C3 fragments and other complement proteins, such as, for example, iC3b, Factor H and the membrane attack complex C5b-C9.

### SUMMARY OF THE CLAIMED INVENTION

The invention provides a chimeric, humanized, veneered or isolated human antibody that preferentially binds to iC3b relative to C3b. Optionally, the antibody is a chimeric, humanized or veneered form of an antibody producible by the method of the present invention. Optionally, the antibody is a monoclonal antibody. Optionally, the antibody binds to an epitope of iC3b within SEQ ID NO:2. Optionally, the antibody binds to an epitope of iC3b within SEQ ID NO:3. Optionally, the antibody binds to a conformational epitope present in iC3b. Optionally, the antibody binds to amyloid plaques in brain tissue of a subject with Alzheimer's disease. Optionally, the antibody binds to drusen. Optionally, the antibody binds to amyloid plaques in the brain tissue of a subject with Alzheimer's disease. Optionally, the antibody is an Fab fragment, single chain Fv, or single domain antibody. Optionally, the isotype is human IgG1.

Optionally, the antibody has at least one mutation in the constant region. Optionally, the mutation reduces complement fixation or activation by the constant region. Optionally, the mutation is at one or more of positions 241, 264, 265, 270, 296, 297, 322, 329 and 331 by EU numbering, for example at positions 318, 320 and 322. Optionally, the isotype is human IgG2 or IgG4 isotype.

Optionally, the antibody is cross-reactive with a non-human iC3b, e.g., a murine iC3b.

Optionally, the antibody is a humanized version of a murine antibody, wherein the murine antibody has a K_{D} at least 10-fold lower for iC3b relative to C3 as determined by surface plasmon resonance. Optionally, the antibody is a humanized version of a murine antibody, wherein the murine antibody has a K_{D} at least 2-fold lower for iC3b relative to C3b or C3 as determined by a sandwich ELISA. Optionally, the antibody is a humanized version of a murine antibody, wherein the murine antibody has at least five-fold greater affinity for iC3b relative to C3 as determined by immunoprecipitation. Optionally, the antibody is a humanized version of a murine antibody, wherein the murine antibody has at least five-fold greater affinity for iC3b relative to C3b as determined by immunoprecipitation.

The invention further provides a pharmaceutical composition comprising any of the above antibodies and a pharmaceutically acceptable excipient.

The invention further provides a method of treating or effecting prophylaxis of a disease characterized by abnormal levels or distribution of iC3b relative to healthy individuals. The method comprises administering an effective regime of an antibody that preferentially binds to iC3b relative to C3b or an agent that induces such an antibody to a patient having or at risk of a disease associated with iC3b aggregation and thereby treating or effecting prophylaxis of the disease. Optionally, the disease is rheumatoid arthritis, systemic lupus erythematosus, acute respiratory distress syndrome (ARDS), a macular degenerative disease, a complement-associated eye condition, age-related macular degeneration, choroidal neovascularization, uveitis, an ischemia-related retinopathy, a diabetic retinopathy, endophthalmitis, diabetic macular edema, pathological myopia, von Hippel-Lindau disease, histoplasmosis of the eye, Central Retinal Vein Occlusion (CRVO), corneal neovascularization, retinal neovascularization, or Alzheimer's disease.

The invention further provides a method of inhibiting formation of drusen comprising administering an effective regime of an antibody that preferentially binds to iC3b relative to C3b or an agent that induces such an antibody to a patient having or at risk of a disease associated with drusen formation and thereby inhibiting drusen formation in the patient.

The invention further provides a method of inhibiting aggregation of iC3b comprising administering an effective regime of an antibody that preferentially binds to iC3b relative to C3b or an agent that induces such an antibody to a patient having or at risk of a disease associated with iC3b aggregation and thereby inhibiting iC3b aggregation in the patient.

The invention further provides a method of stabilizing a non-toxic conformation of iC3b comprising administering an effective regime of an antibody that preferentially binds to iC3b relative to C3b or an agent that induces such an antibody to a patient having or at risk of a disease associated with iC3b and thereby stabilizing a nontoxic conformation of iC3b.

The invention further provides a method of clearing drusen comprising administering an effective regime of an antibody that preferentially binds to iC3b relative to C3b or an agent that induces such an antibody to a patient having drusen and thereby clearing drusen from the patient.

The invention further provides a method of clearing iC3b comprising administering an effective regime of an antibody that preferentially binds to iC3b relative to C3b or an agent that induces such an antibody to a patient having an abnormally high level of iC3b and thereby clearing iC3b from the patient. Optionally, the disease is age related macular degeneration, or Alzheimer's disease.

The invention further provides a method of treating or effecting prophylaxis of a disease associated with iC3b, comprising administering an effective regime of an antibody that preferentially binds to iC3b relative to C3b or an agent that induces such an antibody to a patient having or at risk of the disease and thereby treating or effecting prophylaxis of the disease. The antibody can be any antibody described above or elsewhere. In some methods, the patient is an ApoE2 carrier.

The invention further provides a method of treating or effecting prophylaxis of age related macular degeneration comprising administering an effective regime of an antibody that preferentially binds to iC3b relative to C3 or an agent that induces such an antibody to a patient having or at risk of the disease and thereby treating or effecting prophylaxis of the disease.

The invention further provides a method of treating or effecting prophylaxis of Alzheimer's disease comprising administering an effective regime of an antibody that preferentially binds to iC3b relative to C3b or an agent that induces such an antibody, to a patient having or at risk of the disease and thereby treating or effecting prophylaxis of the disease. The antibody can be an antibody stains plaques in immunohistochemical analysis of AD brain.

The invention further provides a method of reducing amyloid plaque in an Alzheimer's disease patient comprising administering an effective regime of an antibody that preferentially binds to iC3b relative to C3b or an agent that induces such an antibody to a patient having the disease and thereby treating or effecting prophylaxis of the disease. The antibody can be an antibody stains plaques in immunohistochemical analysis of AD brain.

In some methods, the regime is administered topically, intravenously, intravitreally, orally, subcutaneously, intraarterially, intracranially, intrathecally, intraperitoneally, intranasally or intramuscularly. In some methods, the regime is administered intravitreally. In some methods, the antibody has a K_{D} at least 10-fold lower for iC3b than C3b in a Biacore assay. In some methods, the antibody has a K_{D} at least 2-fold lower for iC3b than C3b in an immunoassay in which the iC3b is indirectly immobilized to a plate via an antibody.

The invention further provides a method of producing a monoclonal antibody that preferentially binds to iC3b over C3b. The method comprises immunizing a mammal with substantially intact iC3b and isolating B-cells producing antibodies; forming immortalized cell lines from the isolated B-cells; and screening the cell lines to identify a cell line producing a monoclonal antibody that preferentially binds to iC3b over C3b. The screening can be performed by an immunoassay in which the iC3b is indirectly immobilized to a plate via an antibody. In some methods, the mammal is a rodent, e.g., a mouse. In some methods, the screening is performed by a Biacore assay. The invention further provides a method of producing a monoclonal antibody that preferentially binds to iC3b over C3b. The method comprises immunizing a non-human animal with substantially intact iC3b and isolating B-cells producing antibodies; forming immortalized cell lines from the isolated B-cells; screening the cell lines to identify a cell line producing a monoclonal antibody that preferentially binds to iC3b over C3b; and producing a humanized, chimeric or veneered form of the antibody. The invention further provides a method of producing a monoclonal antibody that preferentially binds to iC3b over C3b. The method comprises immunizing of a human or non-human animal with human immune system genes with substantially intact iC3b and isolating B-cells producing antibodies; forming immortalized cell lines from the isolated B-cells; and screening the cell lines to identify a cell line producing a monoclonal antibody that preferentially binds to iC3b over C3b.

Some methods further comprise administering the monoclonal antibody to a non-human animal disposed to develop deposits of iC3b, and determining whether the monoclonal antibody inhibits, reduces or delays deposits of iC3b or a consequential sign or symptom of a disease associated with such deposits, thereby identifying a monoclonal antibody for activity against a disease associated with iC3b. Some methods further comprise administering the monoclonal antibody to a transgenic non-human animal disposed to develop a characteristic of Alzheimer's disease, and determining whether the monoclonal antibody affects the extent or rate of development of the characteristic relative to a control transgenic nonhuman animal, thereby identifying a monoclonal antibody for activity against Alzheimer's disease. Some methods further comprise administering the monoclonal antibody to a non-human animal disposed to develop amyloid plaque, and determining whether the monoclonal antibody inhibits, reduces or delays deposits of amyloid plaque or a consequential sign or symptom of a disease associated with such amyloid plaque, thereby identifying a monoclonal antibody for activity reducing amyloid plaques.

The invention further provides a method of screening an agent for activity against a disease associated with iC3b. The method comprises administering the agent to a non-human animal disposed to develop deposits of iC3b, and determining whether the agent inhibits, reduces or delays deposits of iC3b or a consequential sign or symptom of a disease associated with such deposits. The invention further provides a method of screening an agent for activity against Alzheimer's disease. The method comprises administering the agent to a transgenic non-human animal disposed to develop characteristic of Alzheimer's disease, and determining whether the agent affects the extent or rate of development of the characteristic relative to a control transgenic nonhuman animal. The invention further provides a method of screening an agent for activity against Alzheimer's disease. The method comprises administering the agent to a non-human animal disposed to develop amyloid plaque, and determining whether the agent inhibits, reduces or delays deposits of amyloid plaque or a consequential sign or symptom of a disease associated with such amyloid plaque. The agent can be (i) an antibody that preferentially binds to iC3b relative to C3b; or (ii) an agent that induces such an antibody.

The invention further provides a method of determining a level of drusen deposits in a patient. The method comprises administering an antibody that preferentially binds to iC3b relative C3b; and detecting presence of bound antibody in the patient. The presence of bound antibody can be determined by positron emission tomography.

The invention provides a chimeric, humanized, veneered or isolated human antibody that preferentially binds to iC3b relative to C3b. Optionally, the antibody is a monoclonal antibody. Optionally, the antibody binds to a neoepitope at the C-terminus of the N-terminal fragment of the alpha chain of iC3b (in common with the C-terminus of C3d). Optionally, the antibody binds to an epitope of iC3b within SEQ ID NO:2. Optionally, the antibody binds to an epitope including residue 20 of SEQ ID NO:2. Optionally, the antibody binds to an epitope including a free carboxyl group of residue 20 of SEQ ID NO:2. Optionally, the antibody binds to an epitope at the N-terminus of the C-terminal fragment of the alpha chain of iC3b. Optionally, the antibody binds to an epitope of iC3b within SEQ ID NO:3. Optionally, the antibody binds to an epitope of iC3b including residue 1 of SEQ ID NO:3. Optionally, the antibody binds to an epitope of iC3b including a free amino group of residue 1 of SEQ ID NO:3. Optionally, the antibody is an Fab fragment, single chain Fv, or single domain antibody. Optionally, the isotype is human IgG1. Optionally, the antibody has at least one mutation in the constant region.

Optionally, the mutation reduces complement fixation or activation by the constant region. Optionally, the mutation is at one or more of positions 241, 264, 265, 270, 296, 297, 322, 329 and 331 by EU numbering, for example at positions 318, 320 and 322. Optionally, the isotype is human IgG2 or IgG4 isotype.

The invention further provides a pharmaceutical composition comprising one or more of the above-mentioned antibodies and a pharmaceutically acceptable excipient. Optionally, the antibody binds to an epitope at the C-terminus of the N-terminal fragment of the alpha chain of iC3b. Optionally, the antibody binds to an epitope at the N-terminus of the C-terminal fragment of the alpha chain of iC3b.

The invention further provides an isolated fragment of iC3b including 3-10 contiguous residues of SEQ ID NO:2 including residue 20 or 3-10 residues of SEQ ID NO:3 including residue 1. Optionally, the isolated fragment has an amino acid sequence consisting of QLPSR or SEETK. Optionally, the fragment is attached to a GGC linker to form an amino acid sequence CGGQLPSR or SEETKGGC. Optionally, the isolated fragment is linked to a carrier molecule optionally via a spacer that helps elicit antibodies against the fragment.

The invention further provides a pharmaceutical composition comprising one or more of the above fragments and an adjuvant acceptable for administration to humans.

The invention further provides a method of inhibiting aggregation of drusen comprising administering an effective regime of an antibody that preferentially binds to iC3b relative to C3b or an agent that induces such an antibody to a patient having or at risk of a disease associated with iC3b aggregation and thereby inhibiting iC3b aggregation in the patient.

The invention further provides a method of stabilizing a non-toxic conformation of iC3b comprising administering an effective regime of an antibody that preferentially binds to iC3b relative to C3b or an agent that induces such an antibody to a patient having or at risk of a disease associated with iC3b and thereby stabilizing a nontoxic conformation of iC3b.

The invention further provides a method of clearing drusen comprising administering an effective regime of an antibody that preferentially binds to iC3b relative to C3b or an agent that induces such an antibody to a patient having drusen and thereby clearing drusen from the patient. The invention further provides a method of clearing iC3b comprising administering an effective regime of an antibody that preferentially binds to iC3b relative to C3b or an agent that induces such an antibody to a patient having an abnormally high level of iC3b and thereby clearing iC3b from the patient.

The invention further provides a method of treating or effecting prophylaxis of a disease associated with iC3b comprising administering an effective regime of an antibody that preferentially binds to iC3b relative to C3b or an agent that induces such an antibody to a patient having or at risk of the disease and thereby treating or effecting prophylaxis of the disease. The antibody or agent can be any antibody or agent described above or elsewhere. In some methods, the patient is an ApoE2 carrier.

The invention further provides a method of treating or effecting prophylaxis of age related macular degeneration comprising administering an effective regime of an antibody that preferentially binds to iC3b relative to C3b or an agent that induces such an antibody to a patient having or at risk of the disease and thereby treating or effecting prophylaxis of the disease.

The invention further provides a method of screening an agent for activity against a disease associated with iC3b, comprising administering the agent to a non-human animal disposed to develop deposits of iC3b, and determining whether the agent inhibits, reduces or delays deposits of iC3b or a consequential sign or symptom of a disease associated with such deposits, wherein the agent is an antibody that preferentially binds to iC3b relative to C3b or an agent that induces such an antibody.

The invention further provides a method of determining a level of drusen deposits in a patient, comprising administering an antibody that preferentially binds to iC3b relative C3b; and detecting presence of bound antibody in the patient. Optionally, the presence of bound antibody is determined by positron emission tomography.

### BRIEF DESCRIPTION OF THE FIGURES

FIG. 1 shows the sequence of human precursor C3 protein. The beta chain, and N- and C-terminal fragments of the alpha chain present in iC3b are underlined.

FIG. 2 shows proteolytic processing of C3 to iC3b. The thioester bond (bold line) formed between Cys and Glu is shown for native C3 (amino acids of the thioester region are in circles). (1) Activation of native C3 by C3-convertases yields C3a and C3b bound to an acceptor R (here via ester linkage). (2) C3b inactivation by factor I and a cofactor. (3) iC3b is further degraded by factor I and CR1. (4) Acceptor-bound C3dg is trimmed by unspecific plasma proteases to C3d.

FIG. 3 shows the relative binding of antibody A209 to iC3b, C3b and C3 in a sandwich ELISA assay.

FIGS. 4 and 5 show the relative binding of antibodies 2H8, 2A10, 6G1 and 5D2 to iC3b, C3b and C3 in a sandwich ELISA assay.

FIG. 6A shows a gel of the co-immunoprecipitation of both C3 and iC3b with antibodies 1A2, 2A10, 2H8 and 6G1, detected with Abnova rabbit polyclonal antibody. FIG. 6B shows a gel of the co-immunoprecipitation of both C3 and iC3b with antibody 5D2 and, as a control, 0.4 μg of iC3b, C3b, and C3 in separate lanes, detected with the same antibody as in FIG. 6A. FIG. 6C shows Western of iC3b, C3b, and C3 detected with the same antibody as in FIG. 6A.

FIG. 7 shows immunoprecipitation of either C3b or iC3b with antibodies 2A10, 2H8, or 6G1 using Protein G-Sepharose.

FIG. 8 shows immunoprecipitation of either C3b or iC3b with antibodies 2A10, 2H8, or 6G1 using Protein G magnetic beads.

FIG. 9 shows immunohistochemical characterization of iC3b antibodies on brain tissue from a man with Alzheimer's disease.

FIG. 10 shows immunohistochemical staining with various concentrations of antibody 6G1 of brain tissue from a man with Alzheimer's disease.

FIG. 11 shows the results of preabsorbing 6G1 with iC3b, C3 or C3b protein prior to immunohistochemical staining of brain tissue from a man with Alzheimer's disease.

FIG. 12 shows the relative binding of 5D2, 2H8, 2A10 and 6G1 to murine iC3b in a sandwich ELISA assay.

FIG. 13A shows Western of iC3b incubated with biotinylated antibody A209 in the absence or presence of 10 μg/ml of competing antibodies (6G1, 2H8, 2A10, 5D2, 1A2, or MAB1-82814). FIGS. 13B and 13C show the binding of biotinylated antibody A209 to iC3b in the presence of competiting antibodies (6G1, 2H8, 2A10, 5D2, 1A2, or MAB1-82814) in a direct ELISA assay.

### BRIEF DESCRIPTION OF THE SEQUENCES

SEQ ID NO:1 is the C3 precursor sequence.

SEQ ID NO:2 is the last 20 amino acids of the N-terminal fragment of the alpha chain in human iC3b.

SEQ ID NO:3 is the first 20 amino acids of the C-terminal fragment of the alpha chain in human iC3b.

SEQ ID NO:4 is the N-terminal fragment of the alpha chain in human iC3b.

SEQ ID NO:5 is C-terminal fragment of the alpha chain of human iC3b.

SEQ ID NO:6 is the tripeptide linker for attaching a carrier to an immunogen.

SEQ ID NO:7 is the last 20 amino acids of the N-terminal fragment of the alpha chain in mouse iC3b.

SEQ ID NO:8 is the first 20 amino acids of the C-terminal fragment of the alpha chain in mouse iC3b.

SEQ ID NO:9 is the sequence of amino acids 1293-1315 of C3 Precursor sequence (SEQ ID NO:1).

SEQ ID NO:10 is the sequence of amino acids 1313-1328 of C3 Precursor sequence (SEQ ID NO:1).

SEQ ID NO:11 is the sequence of amino acids 667 to 671 of C3 Precursor sequence (SEQ ID NO:1).

SEQ ID NO:12 is the sequence of amino acids 1299-1303 of C3 Precursor sequence (SEQ ID NO:1).

SEQ ID NO:13 is the sequence of amino acids 1321-1325 of C3 Precursor sequence (SEQ ID NO:1).

SEQ ID NO:14 is the sequence of amino acids 1299-1303 of C3 Precursor sequence (SEQ ID NO:1).

SEQ ID NO:15 is the sequence of amino acids 1321-1325 of C3 Precursor sequence (SEQ ID NO:1).

SEQ ID NO:16 is the sequence of amino acids 1321-1325 of C3 Precursor sequence (SEQ ID NO:1) linked to a GGC-OH linker.

### DEFINITIONS

Monoclonal antibodies and other therapeutic agents are typically provided in isolated form. This means that the agent is at least partially separated from the components with which it is naturally associated, if any, and/or is typically at least 50% w/w pure of proteins and other macromolecules arising from its production or purification but does not exclude the possibility that the agent is combined with an excess of pharmaceutical acceptable excipient(s) intended to facilitate its use. Sometimes monoclonal antibodies are at least 60%, 70%, 80%, 90%, 95% or 99% w/w pure of proteins and other macromolecules from production or purification. Often an isolated monoclonal antibody or other therapeutic agent is the predominant macromolecular species remaining after its purification. Optionally, an isolated monoclonal antibody or other therapeutic agent is purified to essential homogeneity meaning that no other macromolecular species form a discrete band on gel analysis.

Antibodies of the invention typically bind to their designated target with an association constant (also known as an affinity constant) of at least 10⁶, 10⁷, 10⁸, 10⁹ or 10¹⁰ M⁻¹. Some such antibodies bind to their target with a K_{D} of 10⁻⁷, 10⁻⁸, 10⁻⁹ or 10⁻¹⁰ M. K_{D} is the reciprocal of association constant. Such binding is specific binding in that it is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that a monoclonal antibody binds one and only one target. An antibody may preferentially bind one target relative to another target if the antibody binds with at least 2-fold, 5-fold, 10-fold or greater affinity constant to the first target relative to the second target, which can be determined, for example, by methods discussed below.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region, means a light chain variable region without the light chain signal peptide. The carboxy-terminal portion of each chain defines a constant region primarily responsible for effector function. A constant region can include any or all of a CH1 region, hinge region, CH2 region and CH3 region.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chains or between different light chains are assigned the same number.

The term “antibody” includes intact antibodies and binding fragments thereof. Typically, fragments compete with the intact antibody from which they were derived for specific binding to the target. Fragments include separate heavy chains, light chains Fab, Fab′, F(ab′)₂, F(ab)c, Fv and single domain antibodies. Single (variable) domain antibodies include VH regions separated from their VL partners (or vice versa) in conventional antibodies (Ward et al., 1989, Nature 341: 544-546) as well as VH regions (sometimes known as VHH) from species such as Camelidae or cartilaginous fish (e.g., a nurse shark) in which VH regions are not associated with VL regions (see, e.g., WO 9404678). Single domain antibodies in which one chain is separated from its natural partners are sometimes known as Dabs and single domain antibodies from Caemelidae or cartilaginous fish are sometimes known as nanobodies. Constant regions or parts of constant regions may or may not be present in single domain antibodies. For example, natural single variable domain antibodies from Camelidae include a VHH variable region, and CH2 and CH3 constant regions. Single domain antibodies can be subject of humanization by analogous approaches to conventional antibodies. The Dabs type of antibodies are usually obtained from antibodies of human origin. NANOBODY types of antibody are of Camelidae or shark origin and can be subject to humanization. Fragments can be produced by recombinant DNA techniques, or by enzymatic or chemical separation of intact immunoglobulins. As well as monospecific antibodies, the term “antibody” also includes a bispecific antibody. A bispecific or bifunctional antibody is an artificial hybrid antibody having two different heavy/light chain pairs and two different binding sites (see, e.g., Songsivilai and Lachmann, Clin. Exp. Immunol. 79:315-321 (1990); Kostelny et al., J. Immunol, 148:1547-53 (1992)).

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996). A “neoepitope” is an epitope that becomes accessible only upon some modification relative to a precursor state, such as cleavage, covalent modification (e.g., phosphorylation) or conformational change.

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50% but preferably 75%, 90% or 99% as measured in a competitive binding assay. Likewise a reference antibody competes with a test antibody if an excess of a reference antibody (e.g., at least 2×, 5×, 10×, 20× or 100×) inhibits binding of the test antibody by at least 50%, 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Two-way competition (reference competes with test and vice versa) indicates a more proximate relationship between epitopes (e.g., same or substantially overlapping epitopes) than one way inhibition (e.g., partially overlapping or proximate epitopes).

The term “patient” includes human and other mammalian subjects that receive either prophylactic or therapeutic treatment.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities for antibodies are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

The term “adjuvant” refers to a compound that when administered in conjunction with an antigen augments and/or redirects the immune response to the antigen, but when administered alone does not generate an immune response to the antigen. Adjuvants can augment an immune response by several mechanisms including lymphocyte recruitment, stimulation of B and/or T cells, and stimulation of macrophages.

An individual is at increased risk of a disease if the subject has at least one known risk-factor (e.g., genetic, biochemical, family history, situational exposure) placing individuals with that risk factor at a statistically significant greater risk of developing the disease than individuals without the risk factor.

The term “symptom” refers to a subjective evidence of a disease, such as altered gait, as perceived by the patient. A “sign” refers to objective evidence of a disease as observed by a physician.

Statistical significance means p≦0.05.

### DETAILED DESCRIPTION OF THE INVENTION

### I. General

The present invention provides antibodies and agents that induce antibodies that preferentially bind to iC3b relative to C3b. These antibodies serve to reduce signs or symptoms of diseases associated with deposits of iC3b, such as AMD or AD. Although an understanding of mechanism is not required for practice of the invention, an antibody may reduce signs and/or symptoms of such a disease as a result of the antibody promoting clearing of iC3b (and/or its further degradation products such as C3d and C3c), or inhibiting the iC3b or further degradation products from inter or intramolecular aggregation, or from binding to other molecules, or by stabilizing a non-toxic conformation, among other mechanisms Clearing of iC3b or its further degradation products can be via phagocytosis or otherwise and can be of deposits or iC3b in free form (e.g., in the blood). Clearing of iC3b can inhibit further deposition and/or reduce existing deposits of drusen of which iC3b is a component. Because the antibody preferentially binds to iC3b over C3b, the toxicity of truncated iC3b can be inhibited without unacceptable reduction of the immunological role of C3b.

Antibodies that preferentially bind to iC3b or agents that can induce such antibodies can be used in methods of treating or effecting prophylaxis of AMD or AD and other diseases associated with the presence of iC3b.

### II. C3 Precursor, C3, C3a, C3b and iC3b

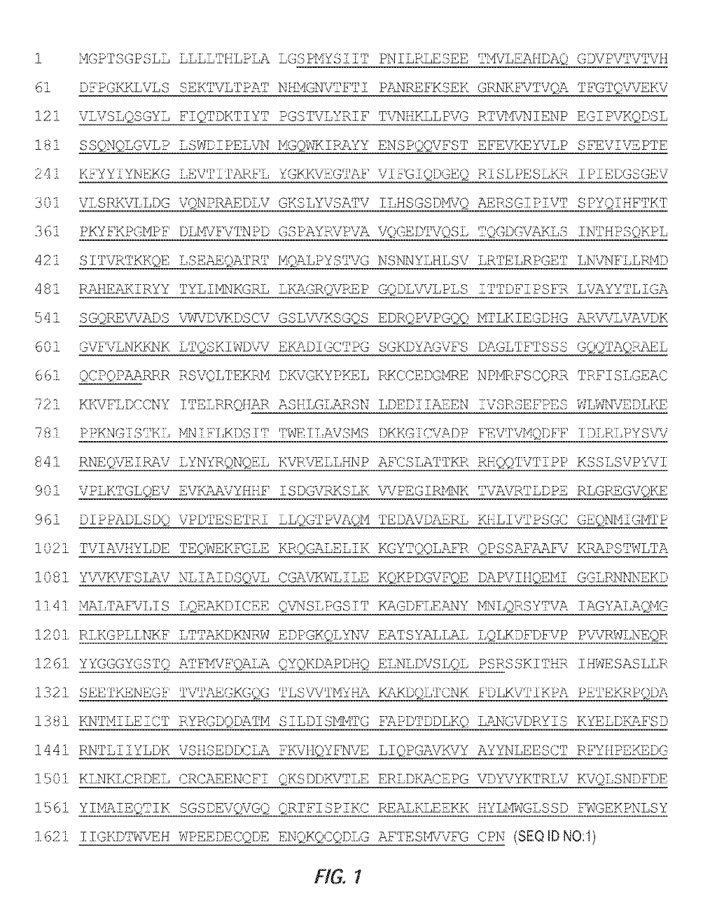

C3, C3a, C3b and iC3b are proteolytic fragments of C3 precursor. Unless otherwise apparent from the context, reference to any of the polypeptides refers to a natural human form thereof. An exemplary human sequence for C3 precursor protein is NCBI P01024.2 or GI:119370332 reproduced in FIG. 1. (The corresponding Swiss Prot identifier is P01024). Natural human variants of this exemplary sequence are also included. Twenty-two such variants are listed in the Swiss-Prot database. Exemplary sequences for C3, C3a, C3b and iC3b can be found as subsequences of the C3 precursor shown in FIG. 1. Amino acids 1-22 of the C3 precursor are a cleaved signal peptide. Amino acids 23-667 form a beta chain. The beta chain is present in each of C3, C3b and iC3b. Amino acids 667 to 671 (RRRR; SEQ ID NO:11) are cleaved in processing of C3 precursor to C3 separating the beta chain from an alpha chain. Residues 672 to 1663 form an alpha chain of C3. Residues 672 to 748 of this alpha chain are cleaved to form the C3a fragment (anaphylatoxin). The remainder of the alpha chain of C3, residues 749 to 1663 forms the alpha chain of C3b. The alpha chain of C3b is cleaved to generate N-terminal and C-terminal fragments and an excised peptide in conversion of C3b to iC3b. The N-terminal fragment runs from residue 749 to 1303 and the C-terminal fragment from residue 1321 to residue 1663. The excised peptide (C3f) not present in iC3b runs from residue 1304 to residue 1320 (annotation of Swiss Prot P01024).

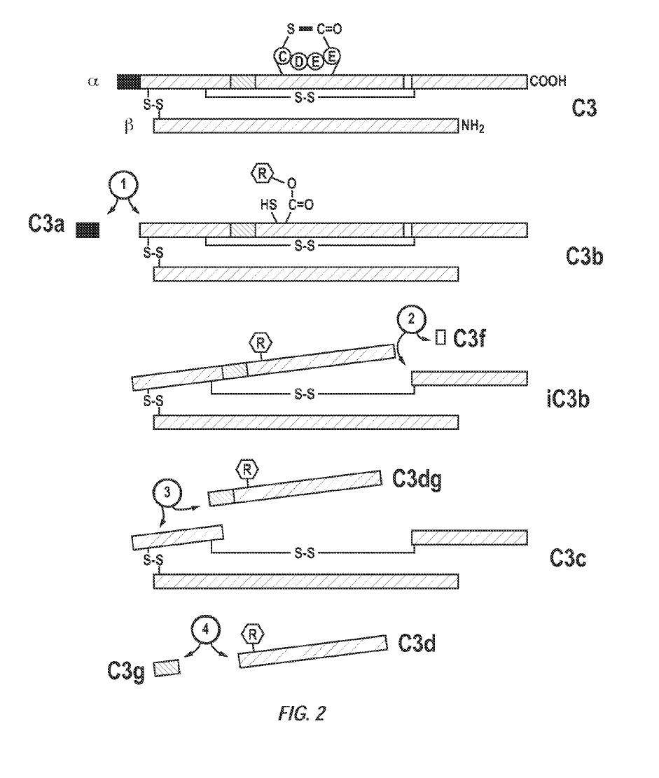

The proteolytic steps in conversion of C3 to iC3b are illustrated by FIG. 2. C3b includes a beta chain and alpha chain held together by disulfide bonding. iC3b constitutes about 0.5% of plasma complement proteins. iC3b differs from C3b in that instead of the complete alpha chain present in C3b, iC3b includes non-contiguous N- and C-terminal fragments held together by disulfide bonding. The different structures of iC3b and C3b give rise to at least two neoepitopes present in iC3b and not present in C3b. One such epitope occurs at the C-terminus of the N-terminal fragment (for example, the arginine residue at position 1303 in the C3 precursor sequence). For ease of reference, the last 20 amino acids of the N-terminal fragment are reproduced and assigned SEQ ID NO:2 (KDAPDHQELN LDVSLQLPSR). This epitope is also present on the further breakdown product C3d (see FIG. 2). The other epitope occurs at the N-terminus of the C-terminal fragment (the N-terminal residue of this fragment being S at position 1321 in the exemplary C3 precursor sequence shown in FIG. 2. The first 20 amino acids of the C-terminal fragment are reproduced and assigned SEQ ID NO:3 (SEETKENEGF TVTAEGKGQG). This epitope is also present on the further breakdown product C3c (see FIG. 2).

The N-terminal fragment of the alpha chain in iC3b has the following sequence (SEQ ID NO:4)

ARASHLGLARSNLDEDIIAEENIVSRSEFPESWLWNVEDLKEPPKNGIS
TKLMNIFLKDSITTWEILAVSMSDKKGICVADPFEVTVMQDFFIDLRLP
YSVVRNEQVEIRAVLYNYRQNQELKVRVELLHNPAFCSLATTKRRHQQT
VTIPPKSSLSVPYVIVPLKTGLQEVEVKAAVYHHFISDGVRKSLKVVPE
GIRMNKTVAVRTLDPERLGREGVQKEDIPPADLSDQVPDTESETRILLQ
GTPVAQMTEDAVDAERLKHLIVTPSGCGEQNMIGMTPTVIAVHYLDETE
QWEKFGLEKRQGALELIKKGYTQQLAFRQPSSAFAAFVKRAPSTWLTAY
VVKVFSLAVNLIAIDSQVLCGAVKWLILEKQKPDGVFQEDAPVIHQEMI
GGLRNNNEKDMALTAFVLISLQEAKDICEEQVNSLPGSITKAGDFLEAN
YMNLQRSYTVAIAGYALAQMGRLKGPLLNKFLTTAKDKNRWEDPGKQLY
NVEATSYALLALLQLKDFDFVPPVVRWLNEQRYYGGGYGSTQATFMVFQ
ALAQYQKDAPDHQELNLDVSLQLPSR

The C-terminal fragment of the alpha chain of iC3b has the following sequence (SEQ ID NO:5)

(SEQ ID NO: 5)
SEETKENEGFTVTAEGKGQGTLSVVTMYHAKAKDQLTCNKFDLKVTIKP
APETEKRPQDAKNTMILEICTRYRGDQDATMSILDISMMTGFAPDTDDL
KQLANGVDRYISKYELDKAFSDRNTLIIYLDKVSHSEDDCLAFKVHQYF
NVELIQPGAVKVYAYYNLEESCTRFYHPEKEDGKLNKLCRDELCRCAEE
NCFIQKSDDKVTLEERLDKACEPGVDYVYKTRLVKVQLSNDFDEYIMAI
EQTIKSGSDEVQVGQQRTFISPIKCREALKLEEKKHYLMWGLSSDFWGE
KPNLSYIIGKDTWVEHWPEEDECQDEENQKQCQDLGAFTESMVVFGCPN

Fragments of iC3b are sometimes referred to by providing a range of the first and last amino acid, as for amino acids 15-20 of SEQ ID NO:2 or 1-5 of SEQ ID NO:3. Such a range defines the start and end point of a fragment but does not preclude the fragment being linked to a heterologous molecule, such as a carrier molecule to form a conjugate. Likewise, antibody binding specificity is sometimes defined by a range of amino acids. If an antibody is said to bind to an epitope within amino acids 15-20 of SEQ ID NO:1, for example, what is meant is that the epitope is within the recited range of amino acids including those defining the outer-limits of the range. It does not necessarily mean that every amino acid within the range constitutes part of the epitope. Thus, for example, an epitope within amino acids 15-20 of SEQ ID NO:2 may consist of amino acids 15-20, 16-19, 17-18, 17-20 or other segments of SEQ ID NO:2.

### III. Antibodies

### A. Binding Specificity and Functional Properties

The invention provides antibodies preferentially binding to iC3b relative to C3b. The antibodies can be monoclonal or polyclonal. Preferential binding means that an antibody binds to iC3b detectably more strongly than to C3b, beyond experimental error, for example with a higher association constant, higher on-rate and/or lower off rate. Some antibodies have affinity constants at least 2, 5 or 10-fold higher for iC3b than C3b. Some antibodies have a K_{D} 2- to 20-fold lower for iC3b than C3b. Some antibodies have an affinity constant at least 10-fold higher for iC3b than C3 or C3b, or a K_{D} at least 10-fold lower for iC3b than C3 or C3b as measured by surface plasmon resonance, for example, in a Biacore assay (e.g., by the procedure of the Examples). Some antibodies have affinity constants at least 2-fold higher for iC3b than C3b as measured in an immunoassay in which the iC3b is indirectly immobilized to a plate via an antibody (e.g., a sandwich ELISA assay, such as described in the Examples). Some antibodies bind to iC3b and lack any significant binding to C3b (i.e., binding indistinguishable between C3b and an irrelevant control protein). Some antibodies preferentially binding to iC3b over C3b are end-specific for the free C-terminus of the C-terminus of the iC3b alpha chain N-terminal fragment and of C3d, such as, for example, the C-terminus of SEQ ID NO:2. Such antibodies may recognize an epitope including the C-terminal amino acid of SEQ ID NO:2 in free form (i.e., with the carboxyl group not attached to any other amino acid as is the case in C3b). End-specific antibodies can bind for example to an epitope within SEQ ID NO:2 or within residues 6-20, 7-20, 8-20, 9-20, 10-20, 11-20, 12-20, 13-20, 14-20, 15-20, 16-20, 17-20, or 18-20 thereof. An example of such a peptide has an amino acid sequence of CGG-QLPSR (SEQ ID NO:14; CGG being a linker).

Some antibodies preferentially binding to iC3b over C3b are end-specific for the free N-terminus of the C-terminal fragment of the alpha chain of iC3b or C3c, such as, for example, the free N-terminus of SEQ ID NO:3 Such end-specific antibodies can bind for example to an epitope within SEQ ID NO:3 or within residues 1-15, 1-14, 1-13, 1-12, 1-11, 1-10, 1-9, 1-8, 1-7, 1-6, 1-5, 1-4 or 1-3 thereof. An example of such a peptide has an amino acid sequence of SEETK-GGC-OH (SEQ ID NO:16; GCC again being a linker).

End-specific antibodies to iC3b showing preferential binding for iC3b relative to C3b may or may not lack any degree of specific binding to C3b (in other words, the preference for iC3b over C3b can be absolute or relative) Other antibodies preferentially binding to iC3b over C3b are not end-specific but may recognize a conformational epitope present on iC3b that is not present or at least not precisely replicated conformationally or thermodynamically in C3b due for example to differences in folding patterns between iC3b and C3b.

End-specific antibodies to the C-terminus of the N-terminal fragment of iC3b (e.g., SEQ ID NO:2) can be generated de novo by immunizing with a peptide including the C-terminal amino acid of this sequence. Usually, such peptides have 3-10 contiguous amino acids from the C-terminus including the C-terminal amino acid, with peptides of 5 or 6 contiguous amino acids being preferred. End-specific antibodies to the N-terminus of the C-terminal fragment of iC3b (e.g., SEQ ID NO:3) can be generated de novo by immunizing with a peptide include the N-terminal amino acid of this sequence. Usually such peptides have 3-10 contiguous amino acids from the N-terminus including the N-terminal amino acid, with peptides of 5 or 6 contiguous amino acids being preferred.

Alternatively, as described in greater detail below, purified iC3b protein or fragments thereof of sufficient length and structure to develop a characteristic conformation (such as the iC3b available from Complement Technology as described in the Examples) or cells, such as sheep red blood cells (SRBC's), containing surface deposited iC3b can be used as an immunogen.

Small peptides are preferably attached to a heterologous carrier molecule forming a conjugate. The carrier molecule helps elicit an antibody response to the peptide. Attachment can be direct or via a spacer peptide or amino acid. Cysteine is used as a spacer amino acid because its free SH group facilitates attachment of a carrier molecule. A polyglycine linker (e.g., 2-6 glycines), with or without a cysteine residue between the glycines and the peptide can also be used. The carrier molecule serves to provide a T-cell epitope that helps elicit an antibody response against the peptide. Several carriers can be used, including keyhole limpet hemocyanin (KLH), ovalbumin and bovine serum albumin (BSA). Peptide spacers can be added to peptide immunogen as part of solid phase peptide synthesis. Carriers are typically added by chemical cross-linking. Some examples of chemical crosslinkers that can be used include cross-N-maleimido-6-aminocaproyl ester or m-maleimidobenzoyl-N-hydroxysuccinimide ester (MBS) (see for example, Harlow, E. et al., Antibodies: A Laboratory Manual, Cold Spring Harbor Laboratory Press, Cold Spring Harbor, N.Y. 1988; Sinigaglia et al., Nature, 336:778-780 (1988); Chicz et al., J. Exp. Med., 178:27-47 (1993); Hammer et al., Cell 74:197-203 (1993); Falk K. et al., Immunogenetics, 39:230-242 (1994); WO 98/23635; and, Southwood et al. J. Immunology, 160:3363-3373 (1998).). When the goal is to generate antibodies to the free C-terminus of a peptide, the carrier and spacer if present are typically attached to the N-terminus of the peptide. Conversely when the goal is to generate antibodies to the free N-terminus of a peptide, the carrier and spacer if present are typically attached to the C-terminus of a peptide. Exemplary protocols for generating end-specific antibodies against other peptides are described by e.g., Konig, Ann NY Acad Sci 777:344-355 (1996), Harrington, Biochim. Biophys. Acta 1158 (2):120-128 (1993); Gravina et al., J. Biol. Chem. 270:(13):7013-6 (1995).

A peptide with optional spacer and carrier or iC3b protein (optionally with spacer and carrier) can be used to immunize laboratory animals or B-cells as described in more detail below. Hybridoma supernatants can be tested for ability to bind the immunogen. The immunogen can be attached to a carrier or other tag to facilitate the screening assay. In this case, the carrier or tag is preferentially different than the combination of spacer and carrier molecule used for immunization to eliminate antibodies specific for the spacer or carrier rather than the iC3b peptide. Antibodies can also be screened against a peptide bridging the site of cleavage generating the neoepitope of the peptide immunogen. For example, if the peptide immunogen ends at residue 20 of SEQ ID NO:2, which corresponds to residue 1303 of SEQ ID NO:1, a peptide bridging the site of cleavage includes at least residues 1303 and 1304 of SEQ ID NO:1 (e.g., NLDVSLQLPSRSSKITHRIHWES. SEQ ID NO:9). Likewise, if the peptide immunogen ends at residue 1 of SEQ ID NO:3, which corresponds to residue 1321 of SEQ ID NO:1, a peptide bridging the site of cleavage includes at least residues 1320 and 1321 of SEQ ID NO:1 (e.g., WESASLLRSEETKENE, SEQ ID NO:10). Antibodies can also be screened for binding to iC3b (using intact or substantially intact iC3b) and for lack of binding or at least reduced binding to C3b. Screening for lack of or reduced binding to C3b (i.e., preferential binding to iC3b relative to C3b) can be performed with C3b itself or a fragment thereof incorporating at least the sequences present in iC3b or a precursor of C3b, such as C3. For such screening assays, the binding target (e.g., iC3b, C3b or C3) can be indirectly immobilized to a plate via an antibody.

The A209 antibody (Quidel) is one example of a mouse monoclonal antibody preferentially binding to iC3b relative to C3b. Another example is the Thermo Scientific antibody MAB1-82814. The present invention provides four additional exemplary mouse monoclonal antibodies designated 6G1, 2A10, 2H8, and 5D2 (produced by hybridomas of the same designation). Each of antibodies 6G1, 2A10, 2H8, or 5D2 preferentially binds to iC3b relative to C3b or C3. For example, each of 6G1, 2A10, 2H8, or 5D2 has an affinity constant at least 2-fold higher for iC3b than C3b as measured by a sandwich ELISA assay, and an affinity constant at least 10-fold higher for iC3b than C3b as measured by a Biacore assay. Antibody 5D2 is cross-reactive between human and mouse iC3b, whereas 6G1, 2A10 and 2H8 specifically bind human iC3b with little if any binding to mouse iC3b. Antibodies 6G1 and 2H8 stain amyloid plaques in brain tissue from an Alzheimer's disease patient, whereas antibodies 2A10 and 5D2 do not. Decreased staining of amyloid plaques with 6G1 was observed when the antibody was pre-absorbed with iC3b (but not with C3b or C3) providing an indication of greater preferential binding of 6G1 to iC3B over C3b or C3.

Some antibodies of the invention bind to the same or overlapping epitope as mouse monoclonal antibodies 6G1, 2A10, 2H8, or 5D2. Some antibodies bind to the same epitope as mouse monoclonal 6G1, 2A10, 2H8, 5D2. Some antibodies compete for specific binding to iC3b with a mouse monoclonal antibody 6G1, 2A10, 2H8, or 5D2. 6G1 and 2H8 do not compete with each other or with A209 or Thermo Scientific antibody MAB1-82814 for specific binding to iC3b (in either one-way or two-way competition analysis) providing an indication each of these three antibodies bind to non-overlapping epitopes. 2A10 and 5D2 do not compete with each other, providing an indication that 2A10 and 5D2 bind to non-overlapping epitopes. 2A10 and 5D2 show a small degree of one-way inhibition in a competition assay with 6G1 or 2H8 indicating that 2A10 and 5D2 bind to distinct epitopes than 6G1 or 2H8 but that the epitopes may be overlapping or proximate to one another.

Antibodies having the binding specificity of a selected murine antibody (e.g. A209, MAB1-82814, 6G1, 2A10, 2H8, and 5D2), and in consequence, sharing at least one, some or all of functional properties of one of the antibodies can be produced by several methods. One such method produces variants of a starting antibody by phage display. See Winter, WO 92/20791. This method is particularly suitable for producing human antibodies. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions can for example be obtained from a library of rearranged human heavy chain variable regions. A phage showing strong specific binding for iC3b (e.g., at least 10⁸ and preferably at least 10⁹ M⁻¹) is selected. The heavy chain variable region from this phage then serves as a starting material for constructing a further phage library. In this library, each phage displays the same heavy chain variable region (i.e., the region identified from the first display library) and a different light chain variable region. The light chain variable regions can be obtained for example from a library of rearranged human variable light chain regions. Again, phage showing strong specific binding for iC3b are selected. The resulting antibodies usually have the same or similar epitope specificity as the murine starting material.

Another method produces variants of designated antibodies by mutagenesis of cDNA encoding the heavy and light chains of an exemplary antibody, such as A209, MAB1-82814, 6G1, 2A10, 2H8, and 5D2. Monoclonal antibodies that are at least 70%, 80%, 90%, 95% or 99% identical to A209, MAB1-82814, 6G1, 2A10, 2H8, or 5D2 in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention. Some antibodies are monoclonal antibodies comprising six CDRs of 6G1, 2A10, 2H8, or 5D2, respectively.

Another method immunizes a non-human animal, such as a mouse, with iC3b or a portion thereof including the desired epitope, and screens resulting antibodies for preferentially binding to iC3b relative to C3b or C3, optionally in competition with a mouse monoclonal antibody 6G1, 2A10, 2H8, or 5D2. Antibodies having the same specificity as one of the exemplified antibodies can also be obtained by immunizing with intact or substantially intact iC3b and screening resulting antibody-producing cells for production of antibodies that compete with one of the exemplified antibodies for binding to iC3b.

Antibodies discriminating between iC3b and C3b that are not end-specific but bind to conformational epitopes present in iC3b but not present or not precisely replicated in C3b can be produced by immunizing with longer peptide immunogens sufficient to develop a characteristic conformation, for example iC3b itself or at least 50, 100, 200 or 250 contiguous residues of one or both of its component chains. Longer peptides can be produced by recombinant expression among other methods. Antibodies generated by such methods are screened for preferential binding to iC3b relative to C3b.

Some antibodies that preferentially bind to iC3b relative to C3b or C3 stain iC3b deposits present in drusen and/or amyloid plaques, e.g., amyloid plaques in brain tissue of a subject with Alzheimer's disease or from a brain of transgenic mouse model thereof. Amyloid plaques are insoluble protein aggregates formed extracellularly by the accumulation of amyloid peptides such as Aβ-42. Amyloid plaque deposits comprise a central core of amyloid fibrils surrounded by dystrophic neuritis, axonal terminals and dendrites, microglia and fibrous astrocytes. As detailed in the Examples, antibodies that stain amyloid plaques can be screened in an immunohistochemical assay of AD brain. Antibodies can be screened for staining drusen by comparing staining of eyes from an AMD mouse model such as any of the models described in greater detail below (for example, ApoE4-HFC mice) and/or human tissue obtained from normal and AMD eyes obtained, for example, from the North Carolina Eye Bank, the Lions Eye Bank, and the Medical Eye Bank of Florida (Ding et al., PNAS 2011; Ramkumar et al., Progress in Retinal and Eye Research 29 (2010) 169-190).

Some antibodies that preferentially bind to iC3b relative to C3b reduce amyloid plaque burden. Antibodies reducing amyloid plaque burden can be screened, e.g., in vivo in animal models or in vitro using a tissue sample from a brain of a patient with Alzheimer's disease or an animal model having characteristic Alzheimer's pathology.

Some antibodies that preferentially bind to iC3b relative to C3b bind to and/or reduce drusen deposits. Antibodies reducing drusen deposits can be screened, e.g., in vivo in animal models or in vitro using a tissue sample from an eye of a patient with AMD or an animal model having characteristic AMD pathology.

Some antibodies (e.g., 6G1, 2A10, 2H8) that preferentially bind to iC3b relative to C3b or C3 bind to human iC3b without significantly binding to iC3b from non-human species (i.e., binding to the non-human species is similar to that of an irrelevant control antibody). Some antibodies (e.g., 5D2) that preferentially bind to iC3b relative to C3b or C3 bind to human iC3b and are cross-reactive with iC3b (but not C3b) from at least one non-human mammalian species. For example, the cross-reactive antibody binds to human iC3b and also binds to iC3b from a non-human primate (e.g., cynomolgus macaque, rhesus macaque, ape, baboon, chimpanzee, orangutan, or gorilla), a rodent (e.g., mouse, rat, hamster, Guinea pig, or rabbit), cow, goat, donkey, pig, dog, cat, or horse. Cross-reactive antibodies have affinity constants for human iC3b within a factor of 2 or 5 for non-human iC3b. Antibodies cross-reacting with rodent iC3b (e.g., murine iC3b) are advantageous in preclinical studies.

### B. Non-Human Antibodies

The production of other non-human monoclonal antibodies, e.g., murine, guinea pig, primate, rabbit or rat, against an immunogen can be performed by, for example, immunizing the animal with an immunogen as described above. See Harlow & Lane, Antibodies, A Laboratory Manual (CSHP NY, 1988) (incorporated by reference for all purposes). Such an immunogen can be obtained from a natural source, by peptide synthesis or by recombinant expression.

Antibodies that preferentially bind to iC3b over C3b can be prepared by immunizing a non-human animal, such as a rodent, including mouse, rat, guinea pig or rabbit, camelid, or cartilaginous fish, with substantially intact iC3b. Immunization can also be performed in vitro on B-cells isolated from a non-human animal or a human. After immunization of an animal or cells in vitro, B-cells are harvested and immortalized, for example, by forming hybridomas. Hybridoma supernatants are tested for production of antibody with the desired properties, particularly binding to iC3b. Antibodies can also be tested for lack of binding or reduced binding to C3b.

Substantially intact iC3b means intact iC3b or an antigen including at least portions of each of its three component chains held together by disulfide bonds as in intact iC3b. Preferably, at least 50, 100, 200 or 250 contiguous residues of each of its component chains are present including all residues responsible for formation of interchain disulfide bonds in intact iC3b.

Optionally, the immunogen can be administered with an adjuvant. Several types of adjuvant can be used as described below. Complete Freund's adjuvant followed by incomplete adjuvant is one suitable regime for immunization of laboratory animals as is the use of RIBI adjuvant. Rabbits or guinea pigs are typically used for making polyclonal antibodies. Mice are typically used for making monoclonal antibodies. Antibodies are screened for specific binding to iC3b. Optionally, antibodies are further screened for lack of specific binding to C3b or to a peptide of C3b including the site of cleavage of a neoepitope in iC3b as described above. Such screening can be accomplished by determining binding of an antibody to a collection of deletion mutants of and determining which deletion mutants bind to the antibody. Binding can be assessed, for example, by Western blot, FACS™ or ELISA.

### C. Humanized Antibodies

A humanized antibody is a genetically engineered antibody in which the CDRs from a non-human “donor” antibody (e.g., 6G1, 2A10, 2H8, or 5D2) are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. No. 5,859,205 6,881,557, Foote, U.S. Pat. No. 6,881,557). The donor antibody can be any antibody preferentially binding to iC3b relative to C3b, such as, for example, an antibody obtained from the immunization of laboratory animals with the immunogens described above or A209 or MAB1-82814. The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

In some antibodies only part of the CDRs, namely the subset of CDR residues required for binding, termed the SDRs, are needed to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196:901, 1987), by molecular modeling and/or empirically, or as described in Gonzales et al., Mol. Immunol. 41: 863, 2004. In such humanized antibodies at positions in which one or more donor CDR residues is absent or in which an entire donor CR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

The human acceptor antibody sequences can optionally be selected from among the many known human antibody sequences to provide a high degree of sequence identity (e.g., 65-85% identity) between a human acceptor sequence variable region frameworks and corresponding variable region frameworks of a donor antibody chain.

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:
(1) noncovalently binds antigen directly,
(2) is adjacent to a CDR region,
(3) otherwise interacts with a CDR region (e.g. is within about 6 Å of a CDR region), (e.g., identified by modeling the light or heavy chain on the solved structure of a homologous known immunoglobulin chain); and
(4) a residue participating in the VL-VH interface.

Framework residues from classes (1)-(3) as defined by Queen, U.S. Pat. No. 5,530,101 are sometimes alternately referred to as canonical and vernier residues. Framework residues defining canonical class of the donor CDR loops determining the conformation of a CDR loop are sometimes referred to as canonical residues (Chothia and Lesk, J. Mol. Biol. 196, 901-917 (1987), Thornton & Martin J. Mol. Biol., 263, 800-815, 1996). A layer of framework residues that support antigen-binding loop conformations play a role in fine-tuning the fit of an antibody to antigen are sometimes referred to as vernier residues (Foote & Winter, 1992, J. Mol. Bio. 224, 487-499). Other candidates for substitution are residues creating a potential glycosylation site. Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position.

### D. Chimeric and Veneered Antibodies

The invention further provides chimeric and veneered forms of non-human antibodies, such as, for example, an antibody obtained from the immunization of laboratory animals with the immunogens described above or A209 or MAB 1-82814, and particularly 6G1, 2A10, 2H8, and 5D2.

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and can be about two-thirds human sequence contributed by the human constant regions.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions.

### E. Human Antibodies.

Human antibodies against iC3b are provided by a variety of techniques described below. Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991) and phage display methods (see, e.g. Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332. Immunization of a transgenic mouse or B-cells in vitro can be performed as described for non-human antibodies. In addition, human antibodies to iC3b may also be obtained via direct cloning of antibodies from plasma B-cells of human volunteers seropositive for the antigen in question, e.g. iC3b in this instance, as described in Wrammert et al. (2008) Nature 453:667-672 & Kashyap et al. (2008) PNAS 105:5986-5991.

### F. Selection of Constant Region

The heavy and light chain variable regions of chimeric, humanized (including veneered), or human antibodies can be linked to at least a portion of a human constant region. The choice of constant region depends, in part, whether antibody-dependent complement and/or cellular mediated cytotoxicity is desired. For example, human isotopes IgG1 and IgG3 have complement-mediated cytotoxicity whereas human isotypes IgG2 and IgG4 have poor or no complement-mediated cytotoxicity. Light chain constant regions can be lambda or kappa. Antibodies can be expressed as tetramers containing two light and two heavy chains, as separate heavy chains, light chains, as Fab, Fab′, F(ab′)2, and Fv, or as single chain antibodies in which heavy and light chain variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. Reference to a human constant region includes a constant region with any natural allotype or any permutation of residues occupying polymorphic positions in natural allotypes or up to 3, 5 or 10 substitutions for reducing or increasing effector function as described below.

One or several amino acids at the amino or carboxy terminus of the light and/or heavy chain, such as the C-terminal lysine of the heavy chain, may be missing or derivatized in a proportion or all of the molecules.

Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

Because antibodies of the invention are used to treat diseases in which pathology is in part mediated by activated forms of complement, it is preferred to include one more substitutions that reduce complement mediated cytotoxicity. Reduction in complement mediated cytotoxicity can be accomplished with or without reduction in Fc receptor binding depending on the nature of the mutation(s). Antibodies with reduced complement mediated cytotoxicity but little or no reduction in Fc receptor allow a desired effect of Fc-mediated phagocytosis of iC3b without activating complement, which may contribute to side effects. Exemplary mutations known to reduce complement-mediated cytotoxicity in human constant regions include mutations at positions 241, 264, 265, 270, 296, 297, 322, 329 and 331. Mutations in positions 318, 320, and 322 have been reported to reduce complement activation in mouse antibodies. Alanine is a preferred residue to occupy these positions in a mutated constant region. Some exemplary human mutations that have been used include F241A, V264A, D265A, V296A, N297A, K322A, and P331S in human IgG3 and D270A or E, N297Q, K322A, P329A, and P331S in human IgG1. The combination of E318A, K320A, R322A mutations can also be used, particularly in human & mouse IgG1 antibodies, to eliminate C1q binding to the Fc region. Here, as elsewhere, the EU numbering scheme is used for numbering amino acids in the constant region of an antibody.

Substitution at any or all of positions 234, 235, 236 and/or 237 reduce affinity for Fcγ receptors, particularly FcγRI receptor and also reduces complement binding and activation (see, e.g., U.S. Pat. No. 6,624,821 WO/2009/052439). An alanine substitution at positions 234, 235 and 237 reduces effector functions, particularly in the context of human IgG1. Optionally, positions 234, 236 and/or 237 in human IgG2 are substituted with alanine and position 235 with glutamine. (See, e.g., U.S. Pat. No. 5,624,821.) to reduce Fc receptor binding.

Exemplary substitutions for increasing half-life include a Gln at position 250 and/or a Leu at position 428.

### G. Expression of Recombinant Antibodies

Chimeric, humanized (including veneered) and human antibodies are typically produced by recombinant expression. Nucleic acids encoding the antibodies can be codon-optimized for expression in the desired cell-type (e.g., CHO, or Sp2/0). Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Examples of such promoters include CMV (e.g., human, mouse or Chinese hamster), ubiquitin or Chinese hamster elongation factor 1(a) (CHEF). Once the vector has been incorporated into the appropriate host, the host is maintained under conditions suitable for high level expression of the nucleotide sequences, and the collection and purification of the cross-reacting antibodies. The vector or vectors encoding the antibody chains can also contain a selectable gene, such as dihydrofolate reductase or glutamate synthase, to allow amplification of copy number of the nucleic acids encoding the antibody chains.

E. coli is a prokaryotic host that can be used for expressing antibodies, particularly antibody fragments. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilizations

Mammalian cells are a preferred host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines, such as DG44, various COS cell lines, HeLa cells, HEK293 cells, L cells, and non-antibody-producing myelomas including Sp2/0 and NS0. Preferably, the cells are nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from endogenous genes, ubiquitin, CHEF, cytomegalovirus, SV40, adenovirus, bovine papillomavirus, and the like. See, e.g., Co et al., J. Immunol. 148:1149 (1992).

Nucleic acids encoding antibody heavy and light chains can be expressed on the same or different vectors. Having introduced vector(s) encoding antibody heavy and light chains into cell culture, cell pools can be screened for growth productivity and product quality in serum-free media. Top-producing cell pools can then be subjected of FACS-based single-cell cloning to generate monoclonal lines. Specific productivites above 50 pg or 100 pg per cell per day, which correspond to product titers of greater than 7.5 g/L culture, are preferred. Antibodies produced by single cell clones can also be tested for turbidity, filtration properties, PAGE, IEF, UV scan, HP-SEC, carboydrate-oligosaccharide mapping, mass spectrometery, and bining assay, such as ELISA or Biacore. A selected clone can then be banked in multiple vials and stored frozen for subsequent use.

Once expressed, antibodies can be purified according to standard procedures of the art, including protein A capture, column chromatography (e.g., hydrophobic interaction or ion exchange), low-pH for viral inactivation and the like (see generally, Scopes, Protein Purification (Springer-Verlag, NY, 1982)).

Methodology for commercial production of antibodies can be employed, including codon optimization, selection of promoters, transcription elements, and terminators, serum-free single cell cloning, cell banking, use of selection markers for amplification of copy number, CHO terminator, serum free single cell cloning, improvement of protein titers (see, e.g., U.S. Pat. No. 5,786,464, U.S. Pat. No. 6,114,148, U.S. Pat. No. 6,063,598, U.S. Pat. No. 7,569,339, WO2004/050884, WO2008/012142, WO2008/012142, WO2005/019442, WO2008/107388, and WO2009/027471, and U.S. Pat. No. 5,888,809).

### IV. Active Immunogens

An agent may be used for active immunization in a patient to induce an immune response comprising antibodies with the binding characteristics and functional properties described above in connection with passive immunization, such as, for example, an antibody preferentially binding to iC3b over C3b. Agents used for active immunization can be the same types of immunogens used for generating monoclonal antibodies in laboratory animals., such as, for example, a peptide of 3-10 contiguous amino acids from the C-terminus of SEQ ID NO:2 or N-terminus of SEQ ID NO:1 or longer peptide immunogens sufficient to develop a conformation characteristic of iC3b relative to C3b or C3. Some examples of iC3b fragments that can be used include iC3b fragments consisting of residues 18-20, 17-20, 16-20, 15-20, 14-20, 13-20, 12-20 or 11-20 of SEQ ID NO:2, or residues 1-3, 1-4, 1-5, 1-6, 1-7, 1-8, 1-9 or 1-10 of SEQ ID NO:3, or at least 50, 100, 200 or 250 contiguous residues of one or both of iC3b component chains sufficient to develop an iC3b characteristic conformation, or iC3b itself. An example of a small peptide immunogen has an amino acid sequence consisting of CGG-QLPSR (SEQ ID NO:14) or SEETK-GGC (SEQ ID NO:15). The tripeptide GGC (SEQ ID NO:6) is a linker that permits a carrier to be attached to the terminal cysteine residue.

The heterologous carrier and adjuvant, if used may be the same as used for generating monoclonal antibody, but may also be selected for better pharmaceutical suitability for use in humans. Suitable carriers include serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking agents of the invention to an immunostimulatory polymer molecule (e.g., tripalmitoyl-S-glycerine cysteine (Pam₃Cys), mannan (a mannose polymer), or glucan (a β1→2 polymer)), cytokines (e.g., IL-1, IL-1 alpha and β peptides, IL-2, γ-INF, IL-10, GM-CSF), and chemokines (e.g., MIP1-α and β, and RANTES) Immunogens may be linked to the carriers with or without spacers amino acids (e.g., gly-gly). Additional carriers include virus-like particles. Virus-like particles (VLPs), also called pseudovirions or virus-derived particles, represent subunit structures composed of multiple copies of a viral capsid and/or envelope protein capable of self-assembly into VLPs of defined spherical symmetry in vivo. (Powilleit, et al., (2007) PLoS ONE 2(5): e415.) Alternatively, peptide immunogens can be linked to at least one artificial T-cell epitope capable of binding a large proportion of MHC Class II molecules., such as the pan DR epitope (“PADRE”). PADRE is described in U.S. Pat. No. 5,736,142, WO 95/07707, and Alexander J et al, Immunity, 1: 751-761 (1994). Active immunogens can be presented in multimeric form in which multiple copies of an immunogen and/or its carrier are presented as a single covalent molecule.

Fragments are often administered with pharmaceutically acceptable adjuvants. The adjuvant increases the titer of induced antibodies and/or the binding affinity of induced antibodies relative to the situation if the peptide were used alone. A variety of adjuvants can be used in combination with an immunogenic fragment of iC3b, to elicit an immune response. Preferred adjuvants augment the intrinsic response to an immunogen without causing conformational changes in the immunogen that affect the qualitative form of the response. Preferred adjuvants include aluminum salts, such as aluminum hydroxide and aluminum phosphate, 3 De-O-acylated monophosphoryl lipid A (MPL™) (see GB 2220211 (RIBI ImmunoChem Research Inc., Hamilton, Mont., now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, Mass.; now Antigenics, Inc., New York, N.Y.). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Ribi adjuvants are oil-in-water emulsions. Ribi contains a metabolizable oil (squalene) emulsified with saline containing Tween® 80. Ribi also contains refined mycobacterial products which act as immunostimulants and bacterial monophosphoryl lipid A. Another adjuvant is CpG (WO 98/40100). Adjuvants can be administered as a component of a therapeutic composition with an active agent or can be administered separately, before, concurrently with, or after administration of the therapeutic agent.

Analogs of natural fragments of iC3b that induce antibodies against iC3b can also be used. For example, one or more or all L-amino acids can be substituted with D amino acids in such peptides. Also the order of amino acids can be reversed (retro peptide). Optionally a peptide includes all D-amino acids in reverse order (retro-inverso peptide). Peptides and other compounds that do not necessarily have a significant amino acid sequence similarity with iC3b peptides but nevertheless serve as mimetics of iC3b peptides and induce a similar immune response can also be used. Anti-idiotypic antibodies against monoclonal antibodies to iC3b as described above can also be used. Such anti-idiotypic antibodies mimic the antigen and generate an immune response to it (see Essential Immunology, Roit ed., Blackwell Scientific Publications, Palo Alto, Calif. 6th ed., p. 181).

Peptides (and optionally a carrier fused to the peptide) can also be administered in the form of a nucleic acid encoding the peptide and expressed in situ in a patient. A nucleic acid segment encoding an immunogen is typically linked to regulatory elements, such as a promoter and enhancer that allow expression of the DNA segment in the intended target cells of a patient. For example, promoter and enhancer elements from light or heavy chain immunoglobulin genes or the CMV major intermediate early promoter and enhancer are suitable to direct expression in blood cells, as is desirable for induction of an immune response. The linked regulatory elements and coding sequences are often cloned into a vector. Antibodies can also be administered in the form of nucleic acids encoding the antibody heavy and/or light chains. If both heavy and light chains are present, the chains are preferably linked as a single chain antibody. Antibodies for passive administration can also be prepared e.g., by affinity chromatography from sera of patients treated with peptide immunogens.

The DNA can be delivered in naked form (i.e., without colloidal or encapsulating materials). Alternatively a number of viral vector systems can be used including retroviral systems (see, e.g., Lawrie and Tumin, Cur. Opin. Genet. Develop. 3, 102-109 (1993)); adenoviral vectors (see, e.g., Bett et al, J. Virol. 67, 591 1 (1993)); adeno-associated virus vectors (see, e.g., Zhou et al., J. Exp. Med. 179, 1867 (1994)), viral vectors from the pox family including vaccinia virus and the avian pox viruses, viral vectors from the alpha virus genus such as those derived from Sindbis and Semliki Forest Viruses (see, e.g., Dubensky et al., J. Virol. 70, 508-519 (1996)), Venezuelan equine encephalitis virus (see U.S. Pat. No. 5,643,576) and rhabdoviruses, such as vesicular stomatitis virus (see WO 96/34625) and papillomaviruses (Ohe et al., Human Gene Therapy 6, 325-333 (1995); Woo et al, WO 94/12629 and Xiao & Brandsma, Nucleic Acids. Res. 24, 2630-2622 (1996)).

DNA encoding an immunogen, or a vector containing the same, can be packaged into liposomes. Suitable lipids and related analogs are described by U.S. Pat. No. 5,208,036, U.S. Pat. No. 5,264,618, U.S. Pat. No. 5,279,833, and U.S. Pat. No. 5,283,185. Vectors and DNA encoding an immunogen can also be adsorbed to or associated with particulate carriers, examples of which include polymethyl methacrylate polymers and polylactides and poly(lactide-co-glycolides), (see, e.g., McGee et al., J. Micro Encap. 1996).

### V. Screening Methods

Antibodies can be initially screened for the intended binding specificity as has already been described (e.g., preferential binding to iC3b over C3b. Active immunogens can likewise be screened for capacity to induce antibodies with such binding specificity. In this case, an active immunogen is used to immunize a laboratory animal and the resulting sera tested for the appropriate binding specificity. Antibodies can also be tested for ability to bind iC3b deposited on cell surfaces e.g., by FACS and inhibit pigment clumping.

Some screening methods are performed by immunoassay Immunoassays include competitive and non-competitive assay systems using techniques such as surface Plasmon resonance, Biacore analysis, FACS analysis, immunofluorescence, immunocytochemistry, Western blot analysis, radioimmunoassay (RIA), Enzyme-Linked ImmunoSorbent Assays (ELISAs), “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, protein A immunoassay, mass spectrometry, immunoblots, competitive binding assay, bead-based assay, radioimmunoprecipitation assay, colloidal gold assays, lateral flow assay, fluorescence polarization assay, nuclear magnetic resonance, and chemiluminescence assay (see, e.g., Ausubel et al., Editors, 1994-present, Current Protocols in Molecular Biology, John Wiley & Sons, Inc., New York, N.Y.).

As shown in the Examples, the conformational epitope of an antibody that preferentially binds to iC3b relative to C3b or C3 can be lost when the iC3b is directly immobilized to a solid support (e.g., a plate), whereas the conformational epitope can be detected when the iC3b is indirectly immobilized to a solid support. Accordingly, preferred screening methods are immunoassays in which the iC3b is indirectly immobilized to a solid support (e.g., via an antibody). Preferably methods include a sandwich ELISA assay and/or a Biacore assay.

Antibodies with a desired binding specificity can be further tested for capacity to induce phagocytosis of iC3b in an in vitro assay. Such an assay includes deposited iC3b and phagocytic cells as well as an antibody under test. The deposited iC3b can be provided as cells, such as SRBC's having iC3b deposited on the cell surface. The deposited iC3b can also be provided as a tissue sample from a disease characterized by deposits of iC3b, such as a tissue sample from AMD affected eyes. The sample is monitored for a reduction in the level of deposit iC3b relative to a baseline level before supplying antibody and/or relative to a negative control lacking the antibody.

Several mouse models of AMD and other diseases characterized by iC3b deposits have been described and can in principle be used for screening antibodies or peptides that induce such antibodies. Antibodies against human iC3b are preferably first tested for cross-reactivity with corresponding mouse iC3b. The corresponding human and mouse sequences around the neoepitopes are as follows: KDAPDHQELN LDVSLQLPSR (SEQ ID NO:2) (human) and TDVPDHKDLN MDVSFHLPSR (SEQ ID NO:7) (mouse) for the C-terminus of the N-terminal fragment and SEETKENEGF TVTAEGKGQG (SEQ ID NO:3) human and SEETKQNEAF SLTAKGKGRG (SEQ ID NO:8) (mouse) for the N-terminus of the C-terminal fragment. There are four contiguous amino acids in common at the neoepitope for the C-terminus of the N-terminal fragment and five amino acids in common at the neoepitope for the N-terminus of the C-terminal fragment. Depending on the antibody, this may or may not be sufficient for conservation of binding between human and mouse epitopes. Alternatively, a transgenic mouse harboring a human C3 transgene can be used. Other examples of animal models of AMD include a knockout model of factor H (Coffey 2007) and mice have null mutation of a Ccl-2 or Ccr-2 gene. These mice develop characteristic signs and symptoms of AMD, including accumulation of lipofuscin and drusen beneath the retinal pigmented epithelium (RPE), photoreceptor atrophy and choroidal neovascularization (CNV) after six months. Other models of AMD with drusen pathology and positive staining for the iC3b neo-epitope has been reported by Radu et al, 2010 ARVO Ann Conf, abstract D626), and C3c (the further breakdown product of iC3b) have been reported (Ambati, Nat Med. 9, 1390-7 (2003)). Other models are the abca4−/− mouse+light (G. Travis, UCLA); ApoE-mice fed a high fat high cholesterol diet (Dithmar et al, (2000) Invest Opthamol Vis Sci 41:2035-2042); and CEP-MSA immunized C57BL/6 (J. Hollyfield, Cle Clinic), which is C3d+ve. Tables 1 and 2, excerpted from Prog. Retinal Eye Res. 29, 169-190 (2010) show properties of these and other mouse models. Other mouse models of AMD have been reported by Ding, et al. (Proc Natl Acad Sci USA. 108:E279-E287, 2012), Sullivan et al. (J Biol Chem 272:17972-17980, 1997). Primate models of AMD have also been described (see, e.g., Hope et al., Brit. J. Ophthalmol. 76, 11-16 (1992)).

Table 1 presents a summary of the mouse and human genetics of the transgenic, immunologically modified, and naturally occurring dry AMD murine models and related diseases. Table 2 presents a summary of the clinical, biochemical, and retinal pathologic findings in dry AMD murine models.

TABLE 1
Genetics of Dry AMD Models

| | | Human | | | |
|---|---|---|---|---|---|
| | Mouse | Disease | Human | Chromosome | |
| # | Model | Association | Gene | Location | OMIM #. |
| 1 | abcr—/— | Stargardt | ABCA4 | 1p21-p13 | *601691 |
| | | disease | | | |
| 2 | ELOVL4- | Stargardt-3 | ELOVL4 | 6q14 | *605512 |
| | mutant | dominant | | | |
| | | inheritary | | | |
| | | disease | | | |
| 3 | Efemp1^{R345W/R345W} | Doyne | EFEMP1 | 2p16 | *601548 |
| | | honeycomb | | | |
| | | retinal | | | |
| | | dystrophy | | | |
| 4 | Timp3^{S156C/S156C} | Sorsby | TIMP3 | 22q12.1-q13.2 | *188826 |
| | | fundus | | | |
| | | dystrophy | | | |
| 5 | cfh^{—/—} | AMD | CFH | 1q32 | +134370 |
| 6 | ccl2^{—/—} | coronary artery | CCL2 | 17q11.2-q12 | +158105 |
| | | disease, | (MCP1) | | |
| | | tuberculosis | | | |
| 7 | ccr2^{—/—} | atherosclerosis, | CCR2 | 3p21 | *601267 |
| | | rheumatoid | | | |
| | | arthritis | | | |
| 8 | cx3cr1^{—/—} | AMD, | CX3CR1 | 3pter-p21 | *601470 |
| | | coronary artery | | | |
| | | disease, | | | |
| | | HIV | | | |
| 9 | ccl2^{—/—}/cx3cr1^{—/—} | coronary artery | CCL2, | 17q11.2-q12, | +158105, |
| | | disease | CX3CR1 | 3pter-p21 | *601470 |
| 10 | sod1^{—/—} | amyotrophic | SOD1 | 21q22.1 | *147450 |
| | | lateral sclerosis | | | |
| 11 | sod2^{—/—} | Leber's | SOD2 | 6q25.3 | *147460 |
| | | hereditary | | | |
| | | optic | | | |
| | | neuropathy, | | | |
| | | idiopathic | | | |
| | | cardiomyopathy | | | |
| 12 | Neprilysin^{—/—} | Alzheimer's | NEPRILYSIN | 3q21-q27 | *120520 |
| | | disease | | | |
| 13 | mcd/mcd | Neuronal | CTSD | 11p15.5 | *116840 |
| | mice | ceroid | | | |
| | | lipofuscinosis | | | |
| 14 | Cp^{—/—}/ | Acerulo- | CP | 3q23-q24 | *117700 |
| | Heph^{—/Y} | plasmin | | | |
| | | emia | | | |
| 15 | ApoE^{—/—} | Hyperlipo- | APOE | 19q13.2 | +107741 |
| | | proteinemia | | | |
| 16 | APO*E3- | Hyperlipo- | APOE | 19q13.2 | +107741.0015 |
| | Leiden | proteinemia | | | |
| 17 | ApoE4 | Type III/V | APOE | 19q13.2 | +107741.0016 |
| | TR | hyperlipo- | | | |
| | | proteinemia, | | | |
| | | Alzhimer's | | | |
| | | disease | | | |
| 18 | APO | Hypobeta- | APOB | 2p24 | +107730 |
| | B100 | lipoproteinemia | | | |
| 19 | arrd2/ | Progressive | MDM1 | 12q14.3 | *164785 |
| | arrd2 | rod-cone | | | |
| | | degeneration, | | | |
| | | AMD | | | |
| 20 | Mfrp^{rd6} | Retinitis | MFRP | 11q23 | *606227 |
| | | pigmentosa | | | |
| 21 | Nr2e3^{rd7} | Enhanced | NR2E3 | 15q23 | *604485 |
| | | S-cone | | | |
| | | syndrome | | | |
| | | (ESCS) | | | |
| 22 | cpfl3 | Achromotopsia | GNAT2 | 1p13 | +139340 |
| 23 | SAMP | aging | unknown | unknown | unknown |
| 24 | SAMR | aging | unknown | unknown | unknown |

| | Genetic | Mouse | Encoding | |
|---|---|---|---|---|
| # | Modification | gene(s) | protein(s) | Reference(s) |
| 1 | knock-out | Abca4 | RmP | Weng et al., Cell. 98: 13-23, |
| | | | | 1999; Mata et al., |
| | | | | Invest Ophthalmol Vis |
| | | | | Sci.42: 1685-90, 2001 |
| 2 | transgenic | Elovl4 | Elov4 | Karan et al., Proc Natl |
| | | | | Acad Sci USA. |
| | | | | 102: 4164-9, 2005 |
| 3 | transgenic | Efemp1 | Fibulin-3 | Marmorstein et al., Hum |
| | | | | Mol Genet. 16: 2423-32 |
| | | | | 2007; Fu et al., Hum |
| | | | | Mol Genet. 16: 2411-22, |
| | | | | 2007 |
| 4 | knock-in | Timp3 | TIMP3 | Weber et al., Invest |
| | | | | Ophthalmol Vis Sci. |
| | | | | 43: 2732-40, 2002 |
| 5 | knock-out | Cfh | CFH | Coffey et al., Proc Natl |
| | | | | Acad Sci USA. |
| | | | | 104: 16651-6, 2007 |
| 6 | knock-out | Mcp1 | MCP1 | Ambati et al., Nat Med. |
| | | | | 9: 1390-7, 2003 |
| 7 | knock-out | Ccr2 | MCP1 | Ambati et al., Nat Med. |
| | | | receptor | 9: 1390-7, 2003 |
| 8 | knock-out | Cx3cr1 | Fractal- | Combadiere et al., J |
| | | | kine | Clin Invest. 117: 2920-8, |
| | | | receptor | 2007 |
| | | | CX3CR1 | |
| 9 | double | Mcp1, | MCP1, | Tuo et al., Invest |
| | knock-out | Cx3cr1 | CX3CR1 | Ophthalmol Vis Sci. |
| | | | | 48: 3827-36, 2007 |
| 10 | knock-out | Sod1 | SOD1 | Imamura et al., Proc |
| | | | | Natl Acad Sci USA. |
| | | | | 103: 11282-7, 2006 |
| 11 | knock- | Sod2 | SOD2 | Sandbach et al., vest |
| | down | | | Ophthalmol Vis Sci. |
| | | | | 42: 2173-8, 2001; |
| | | | | Justilien et al., Invest |
| | | | | Ophthalmol Vis Sci. |
| | | | | 48: 4407-20, 2007 |
| 12 | knock-out | Neprilysin | Amyloid β | Iwata et al., Science. |
| | | | | 292: 1550-2, 2001 |
| 13 | transgenic | mcd | Cathepsin D | Rakoczy et al., Am J |
| | | | | Pathol. 161: 1515-24, |
| | | | | 2002 |
| 14 | knock-out | Cp, | Ferroxidase | Hahn et al., Proc Natl |
| | | sla | cerulo- | Acad Sci USA. |
| | | | plasmin/ | 101: 13850-5, 2004 |
| | | | hepaestin | |
| 15 | knock-out | ApoE | ApoE | Dithmar et al., Invest |
| | | | | Ophthalmol Vis Sci. |
| | | | | 41: 2035-42, 2000 |
| 16 | transgenic | — | apoE3 | Kliffen et al Br J |
| | | | | Ophthalmol. 84: 1415-9, |
| | | | | 2000 |
| 17 | transgenic | — | apoE4 | Malek et al., Proc Natl |
| | | | | Acad Sci USA. |
| | | | | 102: 11900-5, 2005 |
| 18 | transgenic | — | apoB100 | Espinosa-Heidmann et |
| | | | | al. Invest Ophthalmol |
| | | | | Vis Sci. 45: 260-6 2004; |
| | | | | Callow et al. 1994 |
| 19 | none | Mdm1 | Mdm1 | Chang et al., Hum Mol |
| | | | | Genet. 17: 3929-41, |
| | | | | 2008 |
| 20 | none | Mfrp | MFRP | Kameya et al., Hum Mol |
| | | | | Genet. 11: 1879-86, 2002 |
| 21 | none | Nr2e3 | NR2E3 | Ahkmedov et al., Hahn |
| | | | | et al., Proc Natl Acad |
| | | | | Sci USA. 97: 5551-6, |
| | | | | 2000 |
| 22 | none | Gnat2 | GNAT2 | Chang et al., Invest |
| | | | | Ophthalmol Vis Sci. |
| | | | | 47: 5017-21, 2006 |
| 23 | unknown | Mpmv, | unknown | Takada et al., Nippon |
| | | Pmv | | Ganka Gakkai Zasshi. |
| | | | | 98: 955-61, 1994 |
| 24 | unknown | Pmv-35 | unknown | |

TABLE 2
Retinal pathology of dry AMD models

| | | Clinical Information | | | Biochemical Data | | |
|---|---|---|---|---|---|---|---|
| | Mouse | Age of | Clinical | Type of | ERG | A2E | RPE |
| # | Model | onset | exam | lesion | changes | level | changes |
| 1 | abcr—/— | 44 w | not | — | reduced | elevated | lipofuscin, |
| | | | examined | | a-wave | | melanosomes |
| | | | | | amplitude | | vacuolization |
| 2 | ELOVL4- | 7 mo | retinal | RPE | reduced | elevated | lipofuscin, |
| | mutant | | lesions | atrophy | b-wave | | debris, |
| | | | | | responses | | central |
| | | | | | | | retinal RPE |
| | | | | | | | atrophy |
| 3 | Efemp1^{R345W/R345W} | 4 mo | no lesions | none | normal | not | vacuolization, |
| | | | | | a-waves & | measured | loss of basal |
| | | | | | b-waves | | infoldings |
| 4 | Timp3^{S156C/S156C} | 8 mo | no lesions | none | normal | not | basal layer |
| | | | | | b-waves | measured | disruption |
| 5 | cfh^{—/—} | 2 yrs | retinal | hyper- | reduced | not | lipofuscin, |
| | | | lesions | fluorescent | a-wave & | measured | melanosomes |
| | | | | | b-wave | | disorganized |
| | | | | | amplitudes | | organelles |
| 6 | Ccl2^{—/—} | 9 mo | retinal | drusenoid | not | elevated | vacuolated, |
| | | | lesions | | performed | | lipofuscin, |
| | | | | | | | menalosomes |
| 7 | Ccr2^{—/—} | 9 mo | retinal | drusenoid | not | elevated | Hypopigmentation, |
| | | | lesions | | performed | | loss of basal |
| | | | | | | | infolding |
| 8 | Cx3cr1^{—/—} | 12 mo | retinal | drusenoid | not | not | Intracellular |
| | | | lesions | | performed | measured | lipid deposits & |
| | | | | | | | phagosomes |
| 9 | Ccl2^{—/—}/Cx3cr1^{—/—} | 4-6 w | retinal | drusenoid | reduced | elevated | Hypopigmentation, |
| | | | lesions | | a-wave & | | vacuolization, |
| | | | | | b-wave | | lipofuscin, |
| | | | | | amplitudes | | melanosome |
| | | | | | | | loss --> |
| | | | | | | | degeneration |
| 10 | Sod1^{—/—} | 7 mo | retinal | drusenoid | not | not | vacuolization, |
| | | | lesions | | performed | measured | degeneration |
| 11 | Sod2 | 4 mo | not | — | reduced | elevated | lipofuscin, |
| | knock- | | examined | | a-wave & | | hypopigmentation, |
| | down | | | | b-wave | | basal lamina |
| | | | | | amplitudes | | deterioration, |
| | | | | | | | vacuolization, |
| | | | | | | | mitochondrial |
| | | | | | | | abnormalities |
| 12 | neprilysin^{—/—} | 27 mo | not | — | not | not | vacuolization, |
| | | | examined | | performed | measured | loss of tight |
| | | | | | | | and adheren |
| | | | | | | | junctions, |
| | | | | | | | distorted |
| | | | | | | | basal |
| | | | | | | | infolding, |
| | | | | | | | degeneration |
| 13 | mcd/mcd | 12 mo | retinal | RPE | reduced | not | hypertrophy, |
| | mice | | lesions | pigmentary | a-wave & | measured | hypopigmentation, & |
| | | | | changes & | b-wave | | attenuation |
| | | | | drusenoid | amplitudes | | |
| | | | | deposits | | | |
| 14 | Cp^{—/—}/Heph^{—/Y} | 5-6 mo | no | none | not | not | lipofuscin, |
| | | | lesions | | performed | measured | lysosomes, |
| | | | | | | | endosomes, & |
| | | | | | | | phagosomes, |
| | | | | | | | hypopigmentation; |
| | | | | | | | RPE |
| | | | | | | | hypertrophy, |
| | | | | | | | hyperplasia |
| | | | | | | | necrosis |
| 15 | ApoE^{—/—} | 2 mo | not | none | not | not | normal |
| | | | examined | | performed | measured | |
| 16 | ApoE4 | 65 wk-127 wks | not | drusenoid | not | not | vacuolization, |
| | TR | | examined | | performed | measured | hyperpigmentation, |
| | | | | | | | hypopigmentation, |
| | | | | | | | atrophy, |
| | | | | | | | disorganized |
| | | | | | | | basal |
| | | | | | | | infoldings |
| | | | | | | | none |
| | | | | | | | detected; |
| 17 | APOE3- | 9 mo | not | — | not | not | however, |
| | eiden | | examined | | performed | measured | unable to |
| | | | | | | | assess |
| 18 | APOB100 | 3 mo | not | — | not | not | occasional |
| | | | examined | | performed | measured | vacuolization |
| 19 | CEP- | 3 mo | retinal | Patchy | not | not | vesiculation, |
| | immunized | after | lesions | reticular | performed | measured | pyknosis, |
| | mouse | immunezation | | changes | | | lysis, & |
| | | | | | | | areas of RPE |
| | | | | | | | loss |
| 20 | arrd2/arrd2 | 4 mo | retinal | RPE | reduced | not | phagosomes, |
| | | | lesions | hyper- | amplitude, | measured | loss of apical |
| | | | | & hypo- | ultimately | | processes, |
| | | | | pigmentation | extinguished | | hypopigmentation, |
| | | | | vessel | waves | | atrophy |
| | | | | attenuation | | | |
| 21 | Mfrp^{rd6} | 3 mo | retinal | drusenoid | reduced | not | lipofuscin |
| | | | lesions | | amplitude, | measured | |
| | | | | | ultimately | | |
| | | | | | extinguished | | |
| | | | | | waves | | |
| 22 | Nr2e3^{rd7} | 1 mo | retinal | drusenoid | reduced a- | not | Apical |
| | | | lesions | | wave and | measured | villi |
| | | | | | b-wave | | retardation |
| | | | | | amplitudes | | |
| 23 | cpfl3 | 8 mo | no | none | reduced | not | none |
| | | | lesions | | photopic | measured | |
| | | | | | response | | |
| 24 | SAMP8 | 8 mo | not | — | not | not | lipofuscin, |
| | | | examined | | performed | measured | swelling |
| | | | | | | | of basal |
| | | | | | | | infoldings, |
| | | | | | | | microvilli |
| | | | | | | | disruption, |
| | | | | | | | severe |
| | | | | | | | degeneration |

| | Pathologic Information | | | |
|---|---|---|---|---|
| | | BM | Photoreceptor | |
| # | Basal deposits | changes | atrophy | CNV |
| 1 | none | thickening | shortening | none |
| | | | of the | |
| | | | PR OS | |
| 2 | present | none | OS disk | none |
| | | | disorganization, | |
| | | | geographic | |
| | | | atrophy | |
| 3 | BlamD | with | none | none |
| | | amorphous | | |
| | | debris | | |
| 4 | present | thickening | none | present |
| 5 | decreased | thinning | disorganized | |
| | | | PR OS | |
| 6 | present | thickening | pyknotic | present |
| | | | pR, | |
| | | | geographic | |
| | | | atrophy | |
| 7 | present | thickening | ONL | present |
| | | | atrophy, | |
| | | | geographic | |
| | | | atrophy | |
| 8 | present | none | outer retinal | only |
| | | | thinning, | after |
| | | | progressive | laser |
| | | | degeneration | injury |
| 9 | present | thickening | OS | present |
| | | | disorganization & | |
| | | | atrophy | |
| 10 | present | thickening | IS & OS | present |
| | | | loss | |
| 11 | BlamD | thickening | disorganized | none |
| | | | PR OS, | |
| | | | inner retinal | |
| | | | thinning | |
| 12 | BlamD & | none | none | none; |
| | BlinD | | | VEGF |
| | | | | up |
| | | | | regulation, |
| | | | | PEDF |
| | | | | down |
| | | | | regulation |
| 13 | BlamD & | none | ONL | none |
| | BlinD | | thinning, | |
| | | | INL | |
| | | | disorganization | |
| 14 | present | vacuolezation | focal areas | present |
| | | | of PR loss, | |
| | | | progressing | |
| | | | to eventual | |
| | | | loss of IS, | |
| | | | OS, & ONL | |
| | | | thinning | |
| 15 | BlinD | vacuolezation | none | none |
| 16 | BlamD | thickening | thinning of | present |
| | | | the ONL | |
| 17 | BlamD | thickening | none | none |
| 18 | BlamD | thickening | none | none |
| 19 | BlamD | thickening | swollen PR | only |
| | | | | after |
| | | | | laser |
| | | | | injury |
| 20 | none | none | INL & ONL | none |
| | | | thinning, | |
| | | | necrotic | |
| | | | synapses, | |
| | | | OS | |
| | | | fragmentation | |
| 21 | none | none | PR degeneration | none |
| | | | in | |
| | | | all layers | |
| 22 | none | none | ONL | none |
| | | | dysplasia | |
| | | | with whorls | |
| | | | and rosettes, | |
| | | | progressive | |
| | | | thinning | |
| 23 | none | none | vacuolezation, | none |
| | | | PR OS | |
| | | | shortening | |
| 24 | BlamD | thickening, | none | present |
| | | fragmentation | | |

Abbreviations:
RPE, retinal pigment epithelium;
CNV, choroidal neovascularization;
ERG, electroretinography;
BM, Bruch's membrane;
mo, month;
A2E, N-retinylidene-N-retinylethanolamine;
BlamD, basal laminar deposits;
BlinD, basal linear deposits;
PR, photoreceptors;
OS, outer segment;
ONL, outer nuclear layer;
IS, inner segment;
VEGF, vascular endothelial growth factor;
PEDF, pigment epithelium-derived factor.

The invention also provides methods of screening an antibody for activity in reducing amyloid plaques or associated biological entity, for which such activity is desired. To screen for activity against amyloid plaques, a tissue sample from a brain of a patient with Alzheimer's disease or an animal model having characteristic Alzheimer's pathology is contacted with phagocytic cells bearing an Fc receptor, such as microglial cells, and the antibody under test in a medium in vitro. The phagocytic cells can be a primary culture or a cell line, such as BV-2, C8-B4, or THP-1. In some methods, the components are combined on a microscope slide to facilitate microscopic monitoring. In some methods, multiple reactions are performed in parallel in the wells of a microtiter dish. In such a format, a separate miniature microscope slide can be mounted in the separate wells, or a nonmicroscopic detection format, such as ELISA detection of Aβ can be used. Preferably, a series of measurements is made of the amount of amyloid plaques in the in vitro reaction mixture, starting from a baseline value before the reaction has proceeded, and one or more test values during the reaction. The antigen can be detected by staining, for example, with a fluorescently labeled antibody to Aβ or other component of amyloid plaques. The antibody used for staining may or may not be the same as the antibody being tested for clearing activity. A reduction relative to baseline during the reaction of the amyloid plaques indicates that the antibody under test has amyloid plaque reducing activity. Such antibodies are likely to be useful in preventing or treating Alzheimer's disease and other amyloidogenic diseases.

Several animal models of Alzheimer's disease and other diseases characterized by amyloid plaques have been described and can be used for screening antibodies clearing amyloid plaques or having activities against Alzheimer's disease. Examples of animal models of Alzheimer's disease include animals that express human familial Alzheimer's disease (FAD) p-amyloid precursor (APP), animals that overexpress human wild-type APP, animals that overexpress p-amyloid 1-42 (pA), animals that express FAD presenillin-1 (PS-1) (see, e. g., Higgins, L S, 1999, Molecular Medicine Today 5: 274-276), mice overexpressing glycogen synthase kinase (GSK) (see Lucas et al, EMBO J. 20, p27-39, 2001), mice overexpressing mutant alleles of APP or PS1, double (APP/PS1) transgenic mouse models overexpressing mutant alleles of both APP and PS1, double transgenic mice resulting from a cross between a mutant APP line Tg2576 and a mutant PS1M146L transgenic line (Holcomb et al., Nat. Med. 4(1):97-100, 1998), transgenic mice over-expressing the “Swedish” mutant amyloid precursor protein (APP; Tg2576; K670N/M671L; Hsiao et al, 1996, Science, 274:99-102), transgenic APPV717F mice (a.k.a. PDAPP mice; Games et al., Nature 373: 523-527, 1995), and a cohort of PDAPP mice lacking apoE (Bales et al., Nat. Genet. 17: 263-64, 1997).

Active immunogens can also be tested for induction of antibodies in the sera. Both passive and active immunogens can be tested for passage of antibodies across the blood brain barrier into the eyes of an animal model. In these experiments, it is not necessary that the antibody bind to a native neoepitope in the animal.

Antibodies or fragments inducing an antibody can also be tested in non-human primates that naturally or through induction develop symptoms of diseases characterized by iC3b, such as AMD, rheumatoid arthritis and SLE.

Tests on an antibody or active agent are usually performed in conjunction with a control in which a parallel experiment is conducted except that the antibody or active agent is absent (e.g., replaced by vehicle). Reduction, delay or inhibition of signs or symptoms disease attributable to an antibody or active agent under test can then be assessed relative to the control.

### VI. Patients Amenable to Treatment

Patients amenable for treatment have or are at risk of developing an iC3b-associated disorder. Such a disorder means a disease characterized by abnormal levels or distribution of iC3b relative to healthy individuals and particularly diseases characterized by extracellular deposits formed by aggregates of iC3b, sometimes in association with other polypeptides and/or lipids. Such deposits stain with an antibody binding to a neoepitope on iC3b or other drusen component, or with. Such deposits are relatively insoluble in water compared with detergents or denaturing agents, such as guanidine. Such disorders may also be associated with elevated levels of iC3b in body fluids, such as plasma of CSF. iC3b-associated disorders include rheumatoid arthritis, systemic lupus erythematosus, acute respiratory distress syndrome (ARDS), macular degenerative diseases and other complement-associated eye conditions. Complement-associated eye conditions include macular degenerative diseases, such as all stages of age-related macular degeneration (AMD), including dry and wet (non-exudative and exudative) forms, choroidal neovascularization (CNV), uveitis, diabetic and other ischemia-related retinopathies, endophthalmitis, and other intraocular neovascular diseases, such as diabetic macular edema, pathological myopia, von Hippel-Lindau disease, histoplasmosis of the eye, Central Retinal Vein Occlusion (CRVO), corneal neovascularization, and retinal neovascularization iC3b is also a component of plaques present in Alzheimer's disease (Loeffler et al., J. Neuroinflamm. 2008 5:9) so Alzheimer's and related diseases, such as mild-cognitive impairment can also be treated by the present methods.

The methods are particularly suitable for treating age-related macular degeneration (AMD). AMD is age-related degeneration of the macula, which is the leading cause of irreversible visual dysfunction in individuals over the age of 60. Two types of AMD exist, non-exudative (dry) and exudative (wet) AMD. The dry, or nonexudative, form involves atrophic and hypertrophic changes in the retinal pigment epithelium (RPE) underlying the central retina (macula), as well as deposits (drusen) on the RPE. Patients with nonexudative AMD can progress to the wet, or exudative, form of AMD, in which abnormal blood vessels called choroidal neovascular membranes (CNVMs) develop under the retina, leak fluid and blood, and ultimately cause a blinding disciform scar in and under the retina. Nonexudative AMD, which is usually a precursor of exudative AMD, is more common. The presentation of nonexudative AMD varies; hard drusen, soft drusen, RPE geographic atrophy, and pigment clumping can be present. Complement components are deposited on the RPE early in AMD and are major constituents of drusen.

Patients amenable to treatment include individuals at risk of disease but not showing symptoms, as well as patients presently showing symptoms. Patients at risk of disease include those having a known genetic risk of a disease. Such individuals include those having relatives who have experienced this disease, and those whose risk is determined by analysis of genetic or biochemical markers. Genetic markers of risk include complement factor H (CFH) polymorphism, which is associated with the risk of an individual to develop AMD and/or CNV. Mutations in CFH can activate complement, which in turn may lead to AMD/CNV. The CFH polymorphism accounts for 50% of the attributable risk of AMD (Klein et al., Science 308:385-9 (2005)). A common haplotype in CFH (HF1/CFH) has been found to predispose individuals to age-related macular degeneration (Hageman et al., Proc. Natl. Acad. Sci. USA, 102(2):7227-7232 (2005)). Other polymorphisms associated with AMD occur in FB, C3 or LOC387715, a tissue protease. ApoE2 is also a genetic marker of risk of AMD and other diseases associated with iCB3. Smoking also confers enhanced risk of AMD.

Individuals having a disease associated with iC3b can usually be identified by conventional criteria. For example, techniques for diagnosing AMD include Fundus Photography and Angiography, Optical Coherence Tomography and Ultrasound Examination and Ultrasound Biomicroscopy.

The presence of an ApoE4 allele has been associated with increased risk, increased severity and/or earlier age of onset of a large number of neurological disease and conditions including Alzheimer's disease (see, e.g., Mayley et al., PNAS 103, 5644-5651 (2006)). Because of the \association between neurological diseases and conditions and an ApoE4 allele, the present regimes can be used in treatment or prophylaxis of any subject that is carrier of an ApoE4 allele having any neurological disease associated with the deposition of iC3b (for example, Alzheimer's disease) or considered at risk of developing one. The present regimes can also be used for treatment or prophylaxis such disease regardless of ApoE4 carrier status. Of the neurological diseases associated with iC3b deposition, the present methods are particularly suitable for treatment or prophylaxis of Alzheimer's disease, and especially in patients who are ApoE4 carriers. Patients amenable to treatment include individuals at risk of Alzheimer's disease but not showing symptoms, as well as patients presently showing symptoms. Patients at risk of Alzheimer's disease include those having a known genetic risk of a disease. Such individuals include those having relatives who have experienced this disease, and those whose risk is determined by analysis of genetic or biochemical markers. Genetic markers of risk include particularly the ApoE4 allele in heterozygous and even more so in homozygous form. Other markers of risk of Alzheimer's disease include mutations in the APP gene, particularly mutations at position 717 and positions 670 and 671 referred to as the Hardy and Swedish mutations respectively, mutations in the presenilin genes, PS1 and PS2, a family history of AD, hypercholesterolemia or atherosclerosis. Individuals presently suffering from Alzheimer's disease can be recognized by PET imaging, from characteristic dementia, as well as the presence of risk factors described above. In addition, a number of diagnostic tests are available for identifying individuals who have AD. These include measurement of CSF tau and A1342 levels. Elevated tau and decreased A1342 levels signify the presence of AD.

In asymptomatic patients, treatment can begin at any age depending on the degree of risk (e.g., 10, 20, 30 years of age) and/or visual confirmation of drusenoid pathology in the eye. Usually, however, it is not necessary to begin treatment until a patient reaches 40, 50, 60 or 70 years of age.

### VII. Pharmaceutical Compositions and Methods of Treatment

In prophylactic applications, an antibody or agent for inducing an antibody or a pharmaceutical composition comprising the same is administered to a patient susceptible to, or otherwise at risk of a disease associated with iC3b (such as, for example, AMD or AD) in a regime (including dose, frequency and/or route of administration) effective to reduce the risk, lessen the severity, or delay the onset of at least one sign or symptom of the disease. In particular, the regime is preferably effective to inhibit or delay accumulation of iC3b in affected tissues, and/or inhibit or delay its toxic effects and/or inhibit and/or delay development of functional deficits (for example, vision in the case of AMD, mobility in the case of rheumatoid arthritis, and cognition or behavior in the case of AD). In therapeutic applications, an antibody or agent to induce an antibody is administered to a patient suspected of, or already suffering from a disease (for example, AMD) in a regime (dose, frequency and route of administration) effective to ameliorate or at least inhibit further deterioration of at least one sign or symptom of the disease. In particular, the regime is preferably effective to reduce or at least inhibit further increase of levels of iC3b, associated toxicities and/or functional deficits.

A regime is considered therapeutically or prophylactically effective if an individual treated patient achieves an outcome more favorable than the mean outcome in a control population of comparable patients not treated by methods of the invention, or if a more favorable outcome is demonstrated in treated patients versus control patients in a controlled clinical trial (e.g., a phase II, phase II/III or phase III trial) at the p<0.05 or 0.01 or even 0.001 level.

Treatment can be monitored in individual patients from conventional signs or symptoms of the disease in question as well as from levels of iC3b either in deposits associated with the disorder or in the blood or other body fluid, such as blood or CSF. A favorable treatment response is indicated by a reduction in iC3b deposits with time or at least inhibition of further increase compared with the increase expected in an otherwise comparable untreated patient. Treatment of eye conditions, such as AMD or CNV, can be monitored by various endpoints commonly used in evaluating intraocular diseases, such as degree or progression of vision loss. Vision loss can be evaluated by measuring the mean change in best correction visual acuity (BCVA) from baseline to a desired time point (e.g., where the BCVA is based on Early Treatment Diabetic Retinopathy Study (ETDRS) visual acuity chart and assessment at a test distance of 4 meters), measuring the proportion of subjects who lose fewer than 15 letters in visual acuity at a desired time point compared to baseline, measuring the proportion of subjects who gain greater than or equal to 15 letters in visual acuity at a desired time point compared to baseline, measuring the proportion of subjects with a visual-acuity Snellen equivalent of 20/2000 or worse at a desired time point, measuring the NEI Visual Functioning Questionnaire, measuring the size of CNV and amount of leakage of CNV at a desired time point, e.g., by fluorescein angiography. Ocular assessments can include performing an eye exam, measuring intraocular pressure, assessing visual acuity, measuring slitlamp pressure, or assessing intraocular inflammation.

Treatment can also be monitored by determining levels of a passively administered or actively induced antibody in the blood or other body fluid of a patient or in a particular body fluid. The level of such antibodies can be determined, for example, by immuno assay, such as ELISA. iC3b or a fragment including a neoepitope thereof can be used as a binding partner in such an assay. However, such a binding partner may also detect antibodies binding to both iC3b and C3b not specific for a neoepitope. Neoepitope specific antibodies can be distinguished from antibodies binding to C3b by any of the methods described above for identifying an antibody that preferentially binds iC3b relative to C3b or C3. Alternatively, in the case of passive administration, a level of an administered antibody to iC3b can be determined using an anti-idiotypic antibody to the administered antibody as a binding partner. Such monitoring is particularly useful for active immunization in assessing when an effective antibody response has developed and if and when a booster immunization is required to restore a waning level of antibody response from a previous immunization.

Effective doses of antibody vary depending on many different factors, including means of administration, target site, physiological state of the patient, whether the patient has a known genetic risk of iC3b associated disease, whether the patient is human or an animal, other medications administered, and whether treatment is prophylactic or therapeutic.

An exemplary dosage range for antibodies is from about 0.01 to 5 mg/kg, and more usually 0.1 to 3 mg/kg or 0.15-2 mg/kg or 0.15-1.5 mg/kg, of patient body weight. Antibody can be administered such doses daily, on alternative days, weekly, fortnightly, monthly, quarterly, or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months.

The amount of an agent for active administration varies from 0.1-500 μg per patient and more usually from 1-100 or 1-10 μg per injection for human administration. The timing of injections can vary significantly from once a day, to once a year, to once a decade. A typical regimen consists of an immunization followed by booster injections at time intervals, such as 6 week intervals or two months. Another regimen consists of an immunization followed by booster injections 1, 2 and 12 months later. Another regimen entails an injection every two months for life. Alternatively, booster injections can be on an irregular basis as indicated by monitoring of immune response.

Antibodies or agents for inducing antibodies can be administered via a peripheral route (i.e., one in which an administered or induced antibody crosses the blood retina barrier to reach an intended site in the eye. Routes of administration include topical, intravenous, intravitreal, oral, subcutaneous, intraarterial, intracranial, intrathecal, intraperitoneal, intranasal or intramuscular. Preferred routes for administration of antibodies are intravenous, subcutaneous and ocular (e.g., eye drops or intravitreal) for ocular disorders Preferred routes for active immunization are subcutaneous and intramuscular. This type of injection is most typically performed in the arm or leg muscles. In some methods, agents are injected directly into a particular tissue where deposits have accumulated.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable excipient, such as a diluent, buffer, stabilizer, salt, sugar, polysorbate or other auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

The present regimes can be administered in combination with another agent effective in treatment or prophylaxis of the disease being treated. For example, in the case of AMD, the present regime can be combined with inhibitors of VEGF, such as bevacizumab, ranibizumab, or aflibercept or in the case of rheumatoid arthritis NSAIDS, corticosteroids, immunosuppresants and TNF-alpha inhibitors, and NSAIDS, corticosteroids, or rituximab for SLE.

All publications (including GenBank Accession numbers, UniProtKB/Swiss-Prot accession numbers and the like), patents and patent applications cited are herein incorporated by reference in their entirety for all purposes to the same extent as if each individual publication, patent and patent application was specifically and individually indicated to be incorporated by reference in its entirety for all purposes. In the event of any variance in sequences associated with Genbank and UniProtKB/Swiss-Prot accession numbers and the like, the application refers to the sequences associated with the cited accession numbers as of the filing date of the application. Unless otherwise apparent from the context, any step, feature, element, embodiment, aspect or the like of the invention can be used in combination with any other. Although the present invention has been described in some detail by way of illustration and example for purposes of clarity and understanding, it will be apparent that certain changes and modifications may be practiced within the scope of the appended claims.

### EXAMPLES

### Example 1

### Immunization with iC3b Fragment SEETKGGC

A Gly, Gly, Cys was added to the C terminal of the SEETK peptide for ease of coupling and conjugated to sheep anti mouse through a maleamide linkage. The injection schedule was as follows.

TABLE 3
AMD-2-SAM injection schedule

| | | Immunization | | | Titers | | |
|---|---|---|---|---|---|---|---|
| Immunogen | | cage | | | to | to | |
| name | peptide | # | Mouse | Adjuvant | peptide | iC3b | Comments |
| AMD-2-SAM | SEETKGGC | 1 | Jax, A/J | RIBI | >200 K | 1 to | |
| | (SEQ ID | | | | | 7 K | |
| | NO: 15) | | | | | | |
| AMD-2-SAM | SEETKGGC | 2 | Jax, A/J | CFA/IFA | 25 to | 1 to | 2 fusions |
| | (SEQ ID | | | | 61 K | 33 K | |
| | NO: 15) | | | | | | |
| AMD-2-SAM | SEETKGGC | 3 | Jax, A/J | RIBI | 39 to | 1 to | cx w/ C3, |
| | (SEQ ID | | | | 161 K | 15 K | C3b, & |
| | NO: 15) | | | | | | iC3b |
| AMD-2-SAM | SEETKGGC | 4 | Jax, A/J | CFA/IFA | 2 to | >17 K | cx w/ C3, |
| | (SEQ ID | | | | 16 K | | C3b, & |
| | NO: 15) | | | | | | iC3b |

Titers to the peptide were high. Titers to iC3b were low and cross reactive to C3 and C3b.

### Example 2

### Immunization with iC3b Fragment CGGQLPSR

Cys, Gly Gly were added to the N terminal of the QLPRS peptide and conjugated as above. The injection schedules are below.

TABLE 4
EPSP.50-SAM injection schedule

| Immunogen | | Immunization | | | Titers | | |
|---|---|---|---|---|---|---|---|
| name | peptide | cage # | Mouse | Adjuvant | to peptide | to iC3b | Comments |
| EPSP.50- | CGGQLPSR | 1 | Jax, A/J | CFA/IFA | >13.00 | | 2 fusions, |
| SAM | (SEQ ID | | | | | | |
| | NO: 14) | | | | | | |
| EPSP.50- | CGGQLPSR | 2 | Jax, A/J | RIBI | 80 to | | 2 fusions |
| SAM | (SEQ ID | | | | 250 K | | |
| | NO: 14) | | | | | | |
| EPSP.50- | CGGQLPSR | 3 | Jax, A/J | RIBI | 24 to | <3 K | 2 fused, 1 |
| SAM | (SEQ ID | | | | 237 K | | frozen |
| | NO: 14) | | | | | | |
| EPSP.50- | CGGQLPSR | 4 | Jax, A/J | RIBI | 15 to | 1 to | cx w/ C3, |
| SAM | (SEQ ID | | | | 333 K | 18 K | C3b & iC3b |
| | NO: 14) | | | | | | |
| EPSP.50- | CGGQLPSR | 5 | Jax, A/J | CFA/IFA | 90 to | 7 to | cx w/ C3, |
| SAM | (SEQ ID | | | | 264 K | 32 K | C3b, & iC3b |
| | NO: 14) | | | | | | |

Titers to the peptide were high; titers to iC3b were low.

### Example 3

### Immunization with iC3b Protein

The injection schedule was as follows:

TABLE 5
iC3b injection schedule

| Immunogen | | Immunization | | | Titers | | |
|---|---|---|---|---|---|---|---|
| Name | peptide | cage # | Mouse | Adjuvant | to peptide | to iC3b | Comments |
| iC3b | protein | 1 | Jax, A/J | RIBI | | 164 to 2,400K | 2 fused, 3 frozen |
| iC3b | protein | 2 | Balb/c | RIBI | | 17 to 419K | 3 frozen |

Fusion JS13: Mouse #1-3 was immunized 6 times, weekly, with 10 μg iC3b/100 μl mixed with RIBI adjuvant via intraperitoneal administration (“IP”). iC3b was obtained from Complement Technology #A115 Tyler, Tex. and EMD Millipore #204863 Darmstadt, Germany. The mouse developed titers 1:405,000 to iC3b. Three days before the fusion, the mouse was boosted with 10 μg iC3b in PBS, half IV & half IP.

Fusion J514: Mouse #1-4 was immunized 4 times, weekly, with 10 μg iC3b/100 μl mixed with RIBI adjuvant, IP. The mouse developed titers 1:2,396,000 to iC3b. 3 days before fusion, the mouse was boosted with 10 μg iC3b in PBS, half via intravenous administration (“IV”) & half IP.

### Example 4

### Characterization of Antibodies by ELISA

Direct ELISA assay format: For primary screening, the plate was coated with EPSP.50-OVA (QLPRS linked to ovabumin). Supernatants from fusion plates, control antibody, immunized mouse bleed, were used as detecting antibodies, respectively. Goat anti-mouse-HRP (Jackson ImmunoResearch #115-035-164) was used as the secondary antibody. 1-Step ABTS (Thermo #37615) was used as the substrate.

For secondary screening, positives from the primary screening were then screened in the sandwich ELISA assay format.

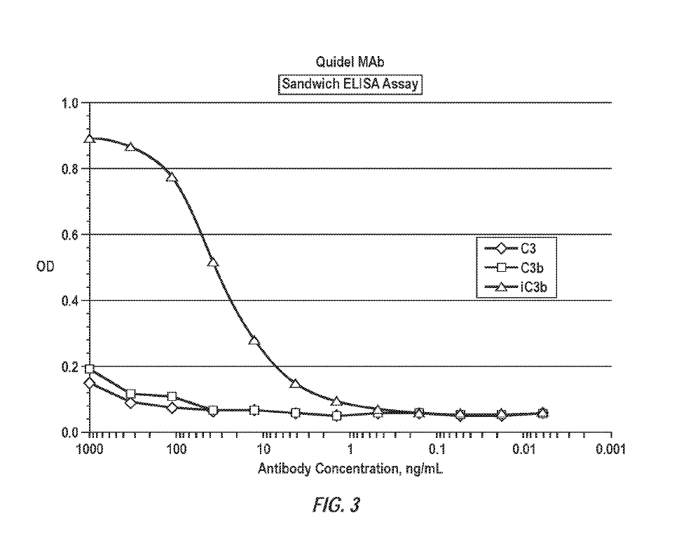

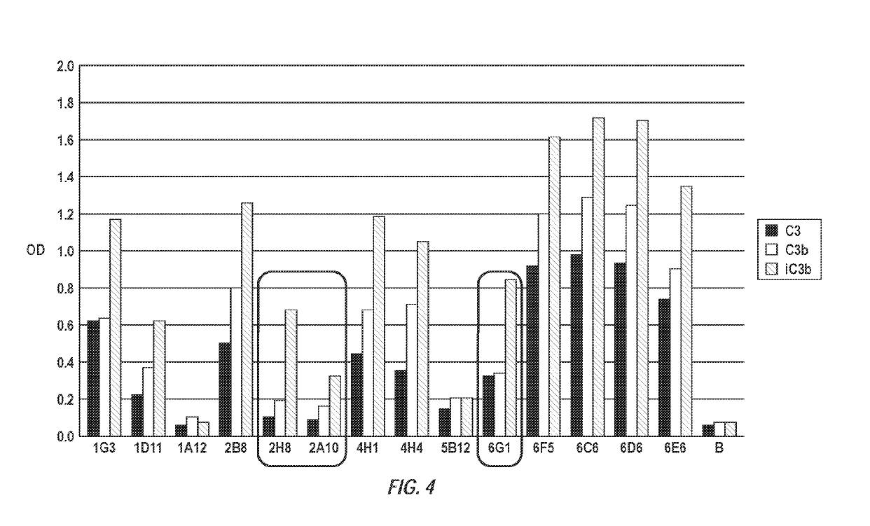

Sandwich ELISA assay format: For secondary screening, C3, C3b and iC3b were used for testing cross-reactivity. The plate was coated with chicken anti-C3 antibody (LeeBio #CC3-80A) as the capture antibody. For primary screening, only iC3b was used for antigen capture. For antibody characterization, all C3 proteins (C3 (Complement Technology #A113); C3b (Complement Technology #A114); and iC3b (Complement Technology #A115)) were used for antigen capture. Supernatants from cells, control antibody, mouse anti-human iC3b (the A209 antibody (Quidel)), were used as detecting antibodies, respectively. Goat anti-mouse-HRP (Jackson ImmunoResearch #115-035-164) was used as the secondary Antibody. 1-Step ABTS (Thermo #37615) was used as the substrate. The control antibody A209 shows preferential binding to iC3b over C3b and C3 (see FIG. 3).

The sandwich ELISA was performed for the two mice in Example 3. First, cell supernatants were only tested for binding to iC3b. C3, C3b, and iC3b were used in the secondary screening for testing cross-reactivity. 2H8, 2A10, 6G1, and 5D2 were shown to preferentially bind iC3b in both ELISA screening and Biacore analysis.

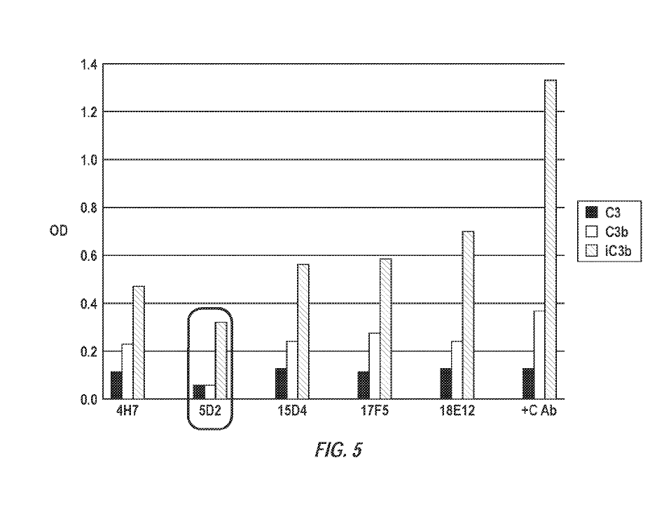

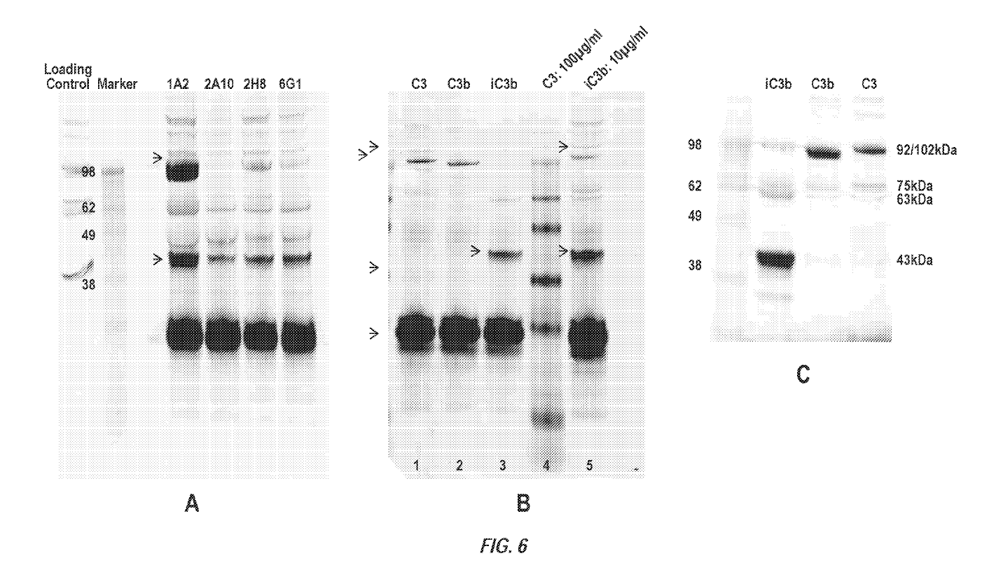

Fusion JS13 (fusion of mouse #1-3 in Example 3) had 96 iC3b positives in primary screen. Fusion JS14 (fusion of mouse #1-4 in Example 3) primary screen had 360 mouse IgG positives in primary screen. Antibodies 2H8, 2A10, 6G1, and 5D2 showed better than 2-fold greater specificity for iC3b vs. C3 or C3b proteins (see FIGS. 4 and 5).

In a direct ELISA with C3, C3b and iC3b coated directly on the plate, all antibodies showed a lack of specificity. This suggests that the antibodies that specifically bound iC3b in the sandwich ELISA may recognize a conformational epitope specific to iC3b that is lost when coated on the plate.

### Example 5

### Characterization of Antibodies by Biacore

A preliminary Biacore assay was done with four concentrations of either iC3b or one of the two potential cross-reactive complement species (e.g., C3b or C3) to assess relative cross-reactivity. Unlike ELISA, a two-fold difference in K_{D} is not sufficient to establish specificity in the Biacore assay. An anti-mouse CM5 chip was prepared following manufacture protocol. Three antibodies (2H8, 2A10, and 6G1) were captured at levels such that Rmax would fall between 25 and 50 RU. Four concentrations of either iC3b or cross-reacting protein were used. Both were from Complement Technologies as described above.

2H8 shows specificity to iC3b relative to C3 (>10× difference in K_{D}) (Table 6). 2A10 shows specificity to iC3b relative to C3 (little binding to C3 under these conditions) (Table 6). 6G1 shows specificity to iC3b relative to C3 (>10× difference in K_{D}) (Table 6).

TABLE 6
Binding kinetic parameters of antibodies 2H8, 2A10, 6G1

| | | Association | Dissociation | Dissociation constant |
|---|---|---|---|---|
| | Antigen | rate (ka) | rate (kd) | (K_{D}) |
| 2H8 | iC3b | 2.9e4 | 4.5e−3 | 153 nM |
| 2H8* | C3 | — | — | >5 μM |
| 2A10** | iC3b | — | — | — |
| 2A10** | C3 | — | — | — |
| 6G1 | iC3b | 9.8e4 | 4.2e−3 | 43.3 nM |
| 6G1 | C3 | 1.3e4 | 2.8e−2 | 2.1 μM |

*Kinetics could not be determined. K_{D} was estimated to be greater than 5 μM (based on observed binding at 500 nM, inferring that actual K_{D} was at least 10-fold above).
**Kinetics cannot be obtained. However, it can be inferred that the affinity of 2A10 for C3 is less than iC3b because although there was not enough binding to iC3b to determine rates, there was no binding at all to C3.

### Example 6

### Characterization of Antibodies by Immunoprecipitation

Co-Immunoprecipitation of both C3 and iC3b with antibodies:

20 μg of C3 and 2 μg of iC3b (Complement Technologies), 5 μg of the test antibodies (1A2, 2A10, 2H8 or 6G1), and 30 μl of washed Protein G-Sepharose (GE Lifesciences) were incubated at 4° C. overnight in 200 μl of phosphated buffered saline (PBS). The precipitates were washed twice with PBS and once with radioimmuoprecipitation assay buffer (RIPA), and the bound protein was eluted by boiling the bead slurry in 20 μl reducing/denaturing sample buffer (Invitrogen). In addition to immunoprecipitation samples, 1 μg C3 and 0.1 μg of iC3b protein was included as a loading control (leftmost lane in FIG. 6A). Samples were resolved by SDS-PAGE on 10% Bis-tris gels (Invitrogen) and transferred to nitrocellulose. After blocking membranes for 1 hr in blocking buffer (Invitrogen), membranes were incubated overnight with rabbit anti-C3 antibody (Abnova, cat#PAB5002) at 0.5 μg/ml, washed with PBST, and incubated with goat anti-rabbit secondary antibody (Licor). Images were captured using a Licor Odyssey scanner. The alpha chain of C3 is about 110 kD in size and the beta chain is about 75 kD in size. The alpha chain of C3b is about 101 kD in size. The N-terminal fragment of the alpha chain of iC3b is about 63 kD in size and the C-terminal fragment of iC3b is about 43 kD in size. 5 μg of 5D2 was incubated with 2 μg of C3, C3b or iC3b (Lanes 1-3 of FIG. 6B) or incubated with 20 μg C3 and 2 μg iC3b (Lane 5 of FIG. 6B) As a control, 0.4 μg of each of iC3b, C3b and C3 protein were run in separate lanes on a gel and detected by the Abnova rabbit polyclonal antibody and GAR-dye (FIG. 6C). As shown in FIG. 6A, a band running between 38 and 49 kD and an equally strong, of not stronger band around 98 kD were detected by the rabbit anti-C3/C3b/iC3b antibody in 1A2 immunoprecipitates, suggesting that 1A2 equivalently pulled down both C3 and iC3b (i.e., does not preferentially recognize iC3b relative to C3). A band running between 38 and 49 kD and a weaker band around 98 kD, suggesting that it preferentially recognizes iC3b relative to C3. As shown in FIGS. 6A and 6B, 6G1, 5D2, and 2A10 also immunoprecipitated iC3b better than C3. Compared to 2H8, the immunoprecipitation with 6G1, 5D2, or 2A10 resulted in a much lighter or undetectable band around 98 kD, indicating that 6G1, 5D2, and 2A10 have an even higher degree of specificity for iC3b.

Immunoprecipitation of Either C3b or iC3b with Antibodies:

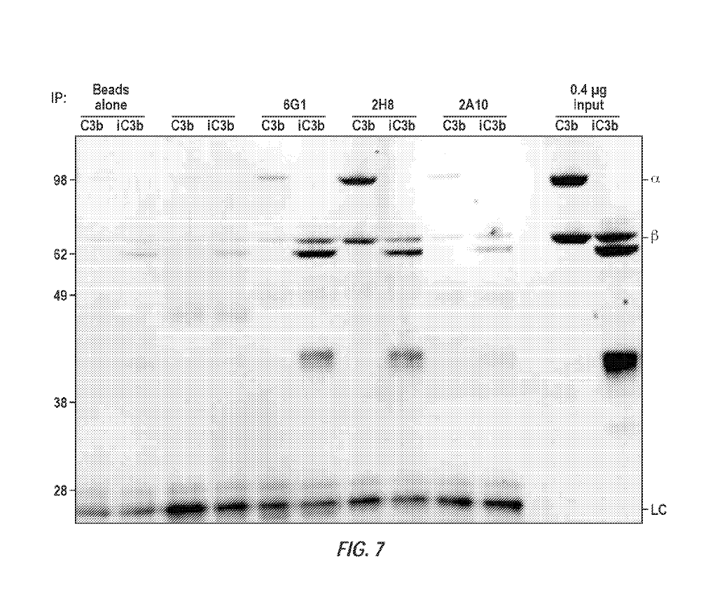

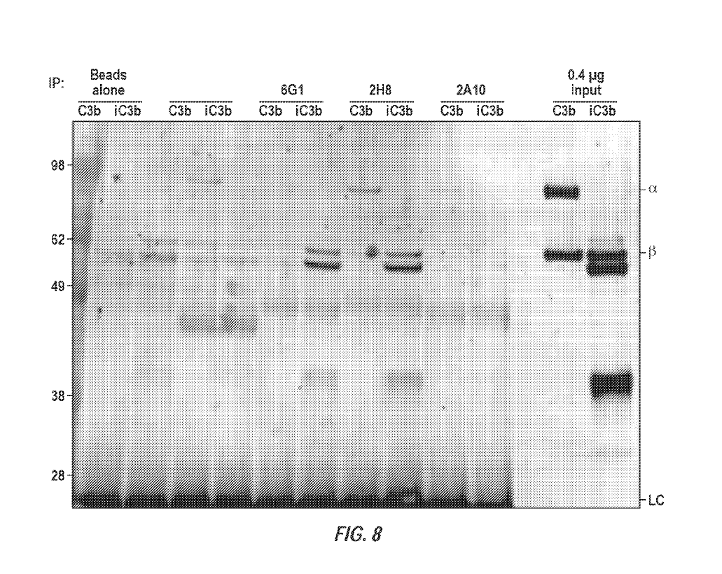

2 μg of either C3b or iC3b (Complement Technologies), 4 μg of the indicated antibodies (2A10, 2H8, and 6G1), and 30 μl of washed Protein G-Sepharose (GE Lifesciences) were incubated at 4° C. overnight in 250 μl of phosphate buffered saline (PBS). The precipitates were washed twice with PBS and once with radioimmunoprecipitation assay buffer (RIPA), and the bound protein was eluted by boiling the bead slurry in 20 μl reducing/denaturing sample buffer (Invitrogen). In addition to immunoprecipitation samples, 0.4 μg each of purified protein was included as a loading control. Samples were resolved by SDS-PAGE on 10% Bis-tris gels (Invitrogen) and transferred to nitrocellulose. After blocking membranes for 1 hr in blocking buffer (Invitrogen), membranes were incubated O/N with rabbit anti-C3 antibody (Abnova, cat#PAB5002) at 0.5 μg/mL, washed with TBST, and incubated with goat anti-rabbit secondary antibody (Licor). Images were captured using a Licor Odyssey scanner. Immunoprecipitation of C3b with an antibody that binds C3b would result in bands at 75 kD and 100 kD in a reducing/denaturing gel Immunoprecipitation of iC3b with an antibody that binds iC3b would result in bands at 43 kD, 63 kD and 75 kD. An antibody specific for iC3b compared to C3b would result in a 43 kD band detected in the iC3b lane and little to no 100 kD band (intact alpha-chain) detected in the C3b lane. As shown in FIG. 7, antibody 6G1 had specificity for iC3b relative to C3b under these conditions. 2H8 also appears to have immunoprecipitated C3b with intact alpha-chain. When Protein G magnetic beads from New England Biolabs were used, 2H8 precipitated less C3b in proportion to iC3b than was observed when the experiment was conducted with Protein G Sepharose® (see FIG. 8).

### Example 7

### Immunohistochemical Characterization of iC3b Antibodies on the Human Alzheimer's Disease Brain

General Protocol: Five iC3b affinity-purified mouse monoclonal antibodies were generated and tested immunohistochemically on minimally fixed frozen sections of human brain cortex from a patient diagnosed with AD and a control. General staining results were shown in Table 7. The Alzheimer's disease sample showed reactivity with some of the antibodies raised against iC3b, staining most prominently in the core of a subset of beta amyloid plaques. The iC3b staining was abundant and mostly confined to the grey matter, with some reactivity detected in the white matter. Normal control sections, in contrast, were largely negative for staining, except for slight reactivity around the vasculature. Of the antibodies that reacted, 6G1 was the most robust and was detected consistently at the core of beta amyloid plaques and showed the most specificity when pre-absorbed with purified human iC3b protein.

TABLE 7
Immunohistochemical characterization of iC3b antibodies

| | | | Stain AD | |
|---|---|---|---|---|
| Antibody | Lot# | Isotype | Tissue | Concentration (mg/ml) |
| 2H8.F6.H7 | NB-0100 | IgG2b/k | Yes | 0.1 μg/mL |
| 6G1.G12.D11 | NB-0101 | IgG2b/k | Yes | 0.1 μg/mL |
| 2A10.D1.F1 | NB-0102 | IgG2b/k | No | — |
| 5D2.B7.D7.B1 | NB-0108 | IgG1/k | No | — |
| 1G3.A1.A3 | NB-0109 | IgG1 + | Yes | 0.1 μg/mL |
| | | IgG2b/k | | |

Methods:

Fresh frozen human brain tissue was obtained from the University of California at San Diego ADRC Brain Bank. Sections of human brain sections were taken from the frontal cortex of 91-year-old male diagnosed with Alzheimer's disease and lacunar infarct (post-mortem interval: unknown; Braak Stage 6.2) and normal cortex from a 77-year old female diagnosed with infarct and acute ischemic changes (PMI: 12 hours; Braak Stage 0). The tissue was cut on a cryostat at 10 μM and mounted directly on charged slides and dried overnight at room temperature. 10 μm, cryocut slide-mounted tissue sections were fixed in acetone (AX0125-4; EMD Chemicals; Gibbstown, N.J.) at −20° C. for 10 minutes. The sections were then rinsed 3×5 minutes in 0.01 M phosphate buffered saline (PBS, pH 7.4; Sigma; P3813-10Pak; St. Louis, Mo.). The sections were then immunohistochemically stained with the various iC3b antibodies at concentrations of 5, 2.5, 1.25, and 0.625 μg/ml. The immunoperoxidase method was the principal detection system, which consisted of a biotinylated goat anti-mouse secondary (JacksonImmuno Research; 115-065-166), a Vector ABC amplification step (ABC Elite Standard; PK-6100; Vector Laboratories), and visualization with a DAB substrate kit (Liquid DAB+Substrate Chromogen System; Dako K3468), which produced a brown deposit. Negative controls consisted of running an IgG-isotype control antibody on serial sections and performing the entire immunohistochemical procedure on adjacent sections in the absence of primary antibody. Tissues were also stained with positive control antibodies (3D6 and GFAP) to ensure that the tissue antigens were accessible for IHC analysis. Slides were imaged with an Olympus Nanozoomer 2.0HT and images were collected as TIFF files.

Pre-Absorptions:

To assess the specificity of the antibodies to its target antigens, the 0.1 μg/mL of the iC3b antibodies were pre-absorbed with 10 μg/mL (100-fold excess) of purified human iC3b, C3, or C3b (Complement Technology, Tyler, Tex.) overnight at 4° C. The antibodies were then applied to tissue and the immunohistochemistry procedure was conducted as outlined above.

Results:

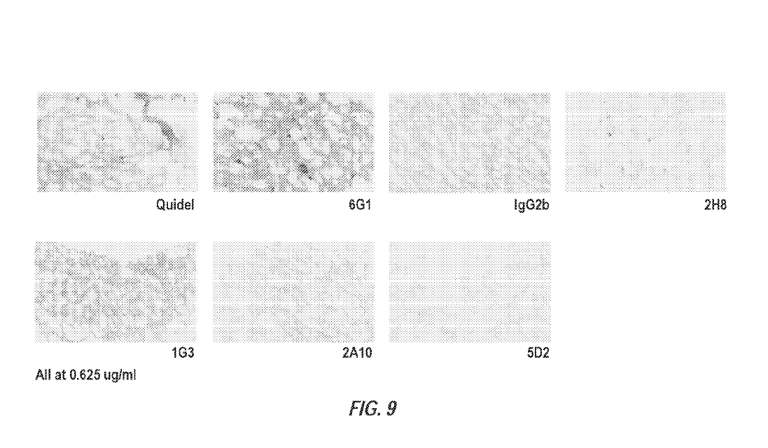

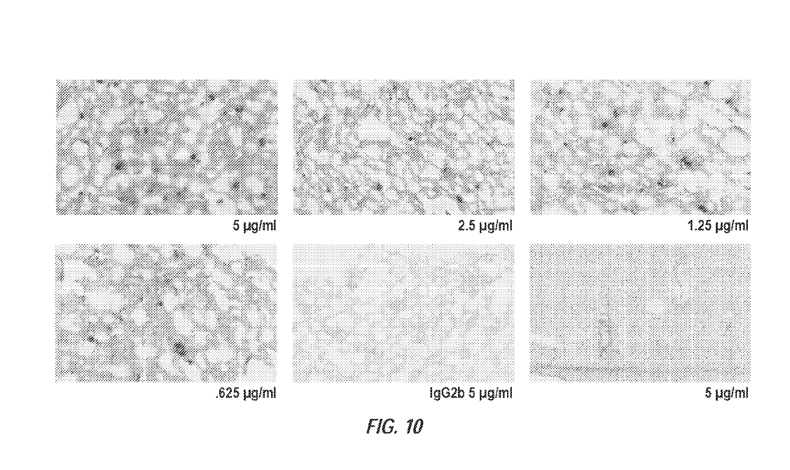

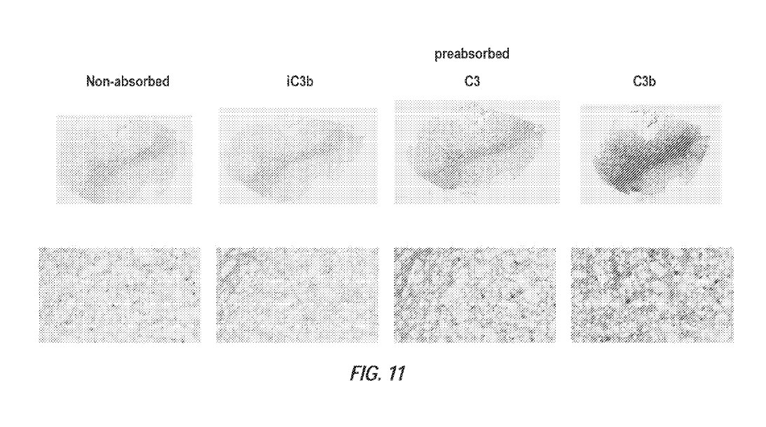

The immunoreactivity of the antibodies that stained the AD brain positively were quite robust at 0.625 μg/mL dilution, labeling plaques with various morphologies in the grey matter (with slight reactivity in the white matter) (FIG. 9). The reference murine monoclonal antibody (Cat. #A209; Quidel Corporation, San Diego, Calif.) showed the least amount of immunoreactivity to plaques when stained in parallel with the other iC3b antibodies at the same concentration. At 0.625 μg/mL dilution, 6G1 was most robust and was therefore further diluted at the sub-microgram levels (FIG. 10). The best signal to noise ratio for 6G1 was attained at 0.1 μg/ml (see, “non-absorbed” tab in FIG. 11).

Pre-absorptions of the various iC3b antibodies with purified human proteins to iC3b, C3b, and C3 showed that the staining was attenuated when 6G1 was pre-absorbed with iC3b. The staining was unaffected when 6G1 was pre-absorbed with C3b or C3 (FIG. 11).

Part I: Primary Antibody Incubation for Slide-Mounted Sections

(1) Block endogenous peroxidase by incubating in 1% hydrogen peroxide in PBS for 30 minutes (H3410-1L; Sigma-Aldrich; St. Louis, Mo.)
(a) Rinse 3×5 minutes in 0.01M PBS, pH 7.4

(2) Incubate slides in with 500 μl of 5% heat-inactivated normal goat serum (#005-000-121; Jackson ImmunoResearch; West Grove, Pa.) in 0.25% Triton X-100 (X100-500 ML; Sigma-Aldrich; St. Louis, Mo.) in 0.01 M phosphate buffered saline for 1 hour at room temperature to block non-specific staining (“5% goat blocking solution”).

(3) Block endogenous biotin in the tissue by incubating in Avidin/Biotin Blocking Kit (SP-2001; Vector Laboratories; Burlingame, Calif.).
(a) Incubate tissue in 250 μl Avidin solution for 15 minutes
(b) Rinse 3×5 minutes in PBS
(c) Incubate tissue in 250 μl Biotin solution for 15 minutes
(d) Rinse 3×5 minutes in PBS

(4) Dilute the primary ic3b antibodies in 5% goat blocking solution according to the concentrations outlined in Table 8 below.

TABLE 8
Recommended dilutions for staining AD brain tissue fixed with acetone

| | | | Stain AD | |
|---|---|---|---|---|
| Antibody | Lot# | Isotype | Tissue | Concentration (mg/ml) |
| 2H8.F6.H7 | NB-0100 | IgG2b/k | Yes | 0.625 μg/mL |
| 6G1.G12.D11 | NB-0101 | IgG2b/k | Yes | 0.100 μg/mL |
| 2A10.D1.F1 | NB-0102 | IgG2b/k | No | No immunoreactivity |
| 5D2.B7.D7.B1 | NB-0108 | IgG1/k | No | No immunoreactivity |
| 1G3.A1.A3 | NB-0109 | IgG1 + | Yes | 0.625 μg/mL |
| | | IgG2b/k | | |

(5) Apply 500 μl of the antibody solution/slide and incubate for 1 hour at room temperature.

Part II: Biotinylated Secondary Antibody and ABC Amplification

(1) Prepare goat anti-mouse secondary antibody.
(a) Dilute goat anti-mouse secondary antibody (“Biotin-SP-AffiniPure Goat Anti-Mouse IgG (H+L) (min X Rat,Hu,Bov,Hrs,Rb Sr Prot); JacksonImmuno Research; 115-065-166) 1:500 in 5% goat blocking solution.

(2) Rinse slides 3× with 0.01M PBS, pH 7.4.

(3) Add 500 μL of secondary antibody solution/slide and incubate for 1 hour at room temperature.

(4) Prepare the ABC solution (ABC Elite Standard; PK-6100; Vector Laboratories, Burlingame, Calif.) by pre-complexing the avidin and biotin solutions 30 minutes prior to its use.
(a) Add 2 drops of A Solution for every 5 mL PBS.
(b) Add 2 drops of B Solution for every 5 mL PBS.
(c) Vortex the solution.

(5) Rinse slides 3×5 minutes in 0.01M PBS, pH 7.4.

(6) Amplify with pre-complexed avid-biotin solution and incubate tissue sections for 1 hour.

(7) Rinse 3×5 minutes in 0.01 M PBS, pH 7.4.

Part III: Visualization with Chromogen

(1) Prepare the ABC solution (ABC Elite Standard; PK-6100; Vector Laboratories, Burlingame, Calif.) by pre-complexing the avidin and biotin solutions 30 minutes prior to its use.
(a) Add 2 drops of A Solution for every 5 mL PBS.
(b) Add 2 drops of B Solution for every 5 mL PBS.
(c) Vortex the solution.

(2) Rinse slides 3×5 minutes in 0.01 M PBS, pH 7.4

(3) Amplify with pre-complexed avid-biotin solution and incubate tissue sections for 1 hour.

(4) Rinse 3×5 minutes in 0.01M PBS, pH 7.4

(5) Prepare the DAB solution (Liquid DAB+Substrate Chromogen System; Dako K3468; Carpinteria, Calif.)
(a) Mix 0.010 mL of Liquid DAB for every 1 mL of DAB substrate (e.g., 0.050 mL for 5 mL DAB substrate solution.
(b) React the tissue sections with 500 μl of chromogen and substrate for 2 minutes or until the appropriate level of staining is attained.

(6) Rinse sections 3×5 minutes each to stop the reaction.

(7) Counterstain the slides in hematoxylin (Modified Harris Hematoxylin; #72704; Richard-Allan Scientific; Kalamazoo, Mich.) then dehydrate in increasing alcohol series (50-, 70-, 95-, 100-, 100-, and 100%), clear in 3 changes of fresh xylene, and coverslip with Cytoseal 60 (#8310-4; Richard-Allan Scientific Kalamazoo, Mich.).

### Example 8

### 5D2 is Cross-Reactive with Murine iC3b

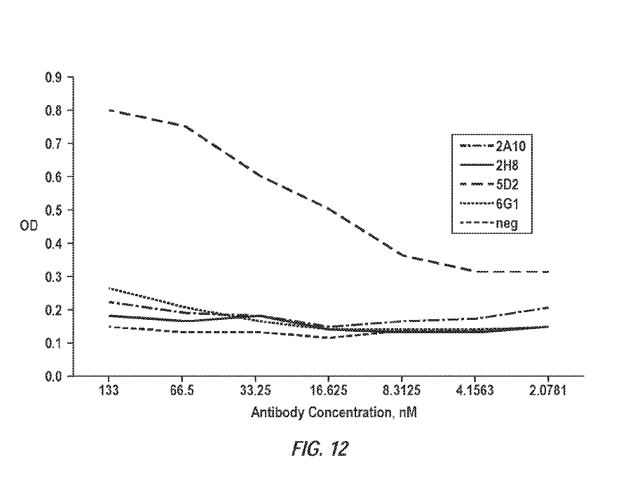

A Costar RIA/EIA (Costar #3590) plate was coated with 5 μg/ml of rat monoclonal antibody 2/11 (Hycult biotech, Cat. HM1065) specific for mouse C3b/iC3b/C3c in 50 μl of PBS per well. The plate was coated at 4° C. overnight. At day 2, the plate was washed 5 times with washing buffer (TPBS+0.05% Tween 20) and blocked with 50 μl blocking buffer (PBS containing 1.5% BSA) per well for 1 hour at room temperature. Then the plate was washed for 5 times with washing buffer and incubated with 50 μl per well of mouse serum diluted with blocking buffer (1:25 dilution). The mouse serum is a serum mixture from two mice. One hour post incubation, the plate was washed for 5 times and incubated with different amount of biotinylated iC3b antibodies as indicated (in 50 μl of blocking buffer). After 1 hour of incubation at room temperature, the plate was washed again for 5 times with washing buffer and incubated with 1: 4000 diluted Streptavidin-HRP (GE Healthcare, RPN 4401V) in 50 μl blocking buffer per well. The plate was washed for 5 times with washing buffer and incubated with 50 μl per well of 1-step ABTS (Thermo Scientific, prod #37615) for 40 minutes at room temperature and read at 405 nM. Mouse serum ELISA data shows that 5D2 cross-reacts with murine iC3b whereas 2H8, 2A10, and 6G1 do not cross-react with murine iC3b (FIG. 12).

### Example 9

### Fortebio-Based Antibody Competition Assay

In order to test whether a first antibody (Ab1) competes with a second antibody (Ab2) for iC3b binding, streptavidin sensor was first dipped in PBS-0.1% BSA for 10 minutes. The sensor was then dipped in PBS-0.1% BSA with various different components in the following order: (1) Step 1: The sensor was dipped in PBS-0.1% BSA for 60 seconds to establish the baseline; (2) Step 2: The sensor was dipped in PBS-0.1% BSA with 5 μg/ml biotinylated Ab1 for 120 seconds to capture Ab1; (3) Step 3: The sensor was dipped in PBS-0.1% BSA containing 100 nM (if Ab1 is 2H8) or 500 nM (if Ab1 is 2A10, 5D2 or 6G1) of purified human iC3b for 120 seconds for the captured Ab1 to bind to iC3b; and (4) Step 4: The sensor was dipped in PBS-0.1% BSA containing 50 μg/ml of Ab2 for 120 seconds to test whether Ab2 can bind to iC3b captured by Ab1.

If the biotinylated Ab1 blocks Ab2 binding, and vice versa (the biotinylated Ab2 also blocks Ab1 binding), it can be concluded that Ab1 and Ab2 bind to the same epitope. If the biotinylated Ab1 doesn't block Ab2 binding and vice versa, it can be concluded that Ab1 and Ab2 bind to different epitope. If the biotinylated Ab1 blocks Ab2 binding but the biotinylated Ab2 does not block Ab1 binding, it can be concluded that Ab1 and Ab2 bind to overlapped epitopes or two epitopes close to each other.

It was found that antibodies 2H8 and 6G1 did not compete with each other, and antibodies 2A10 and 5D2 did not compete with each other. In addition, neither antibody 2H8 nor antibody 6G1 competed with antibody 2A10 or antibody 5D2. However, it was found that antibody 2A10 or antibody 5D2 competes with both antibodies 2H8 and 6G1.

Therefore, antibodies 2H8 and 6G1 bind to different epitopes on iC3b, and antibodies 2A10 and 5D2 bind to different epitopes on iC3b. Antibodies 2A10 and 5D2 bind to epitopes overlapping with or in close proximity to that of 2H8 or 6G1.

### Example 10

### 6G1, 2H8, 2A10, 5D2, and 1A2 do not Compete with A209 and MAB1-82814

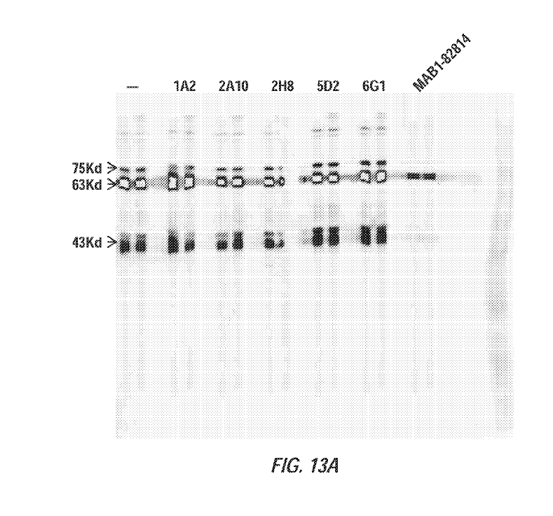

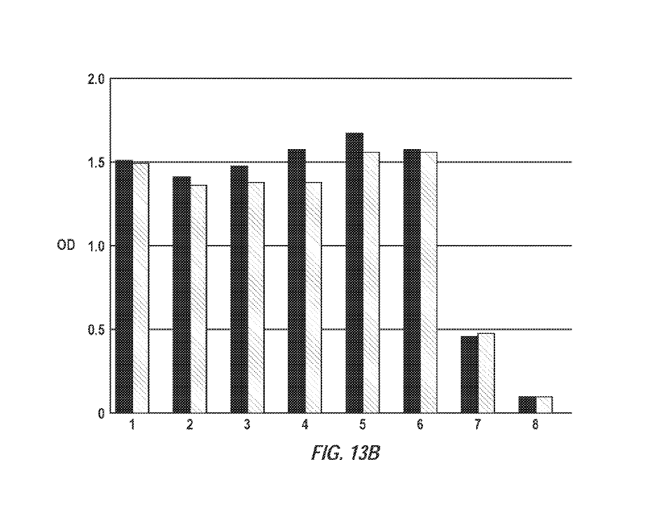

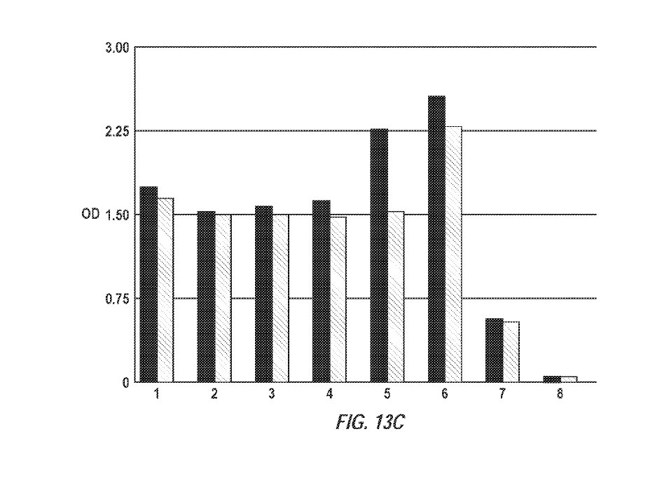

To test whether antibodies 6G1, 2H8, 2A10, 5D2, and 1A2 compete with antibody A209, 0.4 ug of iC3b were resolved by SDS-PAGE on 10% Bis-tris gels (Invitrogen) and transferred to nitrocellulose. After blocking membranes for 1 hour in blocking buffer (Invitrogen), membranes were incubated at room temperature with 1 μg/ml of biotinylated Quidel antibody in the absence or presence of 10 μg/ml of competition Abs as indicated. Membrane was washed with phosphate buffered saline Tween-20 (PBST), and incubated with goat anti-mouse secondary antibody (Licor). Images were captured using a Licor Odyssey scanner. It was found that none of antibodies 6G1, 2H8, 2A10, 5D2, or 1A2 competes with antibody A209 whereas MAB1-82814 competes with A209 (FIG. 13A).

Competition with antibody A209 was also tested using direct ELISA (FIGS. 13B and C). The plate was coated with 10 μg/ml iC3b. Streptavidin-HRP (GE Healthcare, RPN 4401V) was used as detection antibody. In the first ELISA experiment, 1 μg/ml Biotinated antibody A209 and 10 μg/ml of competing MAb (6G1, 2H8, 2A10, 5D2, 1A2, or MAB1-82814) was used as primary antibody (FIG. 13B; Lane 1: 1 μg/ml biotinylated A209; Lane 2: 1 μg/ml biotinylated A209+10 μg/ml 1A2; Lane 3: 1 μg/ml biotinylated A209+10 μg/ml 2A10; Lane 4: 1 μg/ml biotinylated A209+10 μg/ml 2H8; Lane 5: 1 μg/ml biotinylated A209+10 μg/ml 5D2; Lane 6: 1 μg/ml biotinylated A209+10 μg/ml 6G1; Lane 7: 1 μg/ml biotinylated A209+10 μg/ml MAB1-82814; Lane 8: negative control). In the second ELISA experiment, 1 μg/ml Biotinated Quidel MAb and 100 μg/ml of competing MAb (6G1, 2H8, 2A10, 5D2, or 1A2) or 10 μg/ml MAB1-82814 were used as primary antibody (FIG. 13C; Lane 1: 1 μg/ml biotinylated A209; Lane 2: 1 μg/ml biotinylated A209+100 μg/ml 1A2; Lane 3: 1 μg/ml biotinylated A209+100 μg/ml 2A10; Lane 4: 1 μg/ml biotinylated A209+100 μg/ml 2H8; Lane 5: 1 μg/ml biotinylated A209+100 μg/ml 5D2; Lane 6: 1 μg/ml biotinylated A209+100 μg/ml 6G1; Lane 7: 1 μg/ml biotinylated A209+10 μg/ml MAB1-82814; Lane 8: negative control). OD was measured at 405 nm. It was found that none of antibodies 6G1, 2H8, 2A10, 5D2, or 1A2 competes with antibody A209 whereas MAB1-82814 competes with A209 (FIGS. 13B and 13C).

The Western and ELISA results indicated that antibodies 6G1, 2H8, 2A10, 5D2, or 1A2 bind to epitopes different from that of A209 and MAB1-82814.

### Example 11

### Passive Immunization in an APOE4-HFC Mouse Model

APOE4-targeted replacement mice expressing the E4 human apoE isoform were generated as described in Sullivan et al., J Biol Chem 272:17972-17980, 1997. Aged male APOE4 mice (n=104; 65-87 wk) are maintained on a normal rodent chow diet [normal diet (ND), Isopurina 5001; Prolab], and a subset of these mice are switched to an HFC diet (n=84; TD 88051; Harlan Teklad) for 8 wk. The APOE4-HFC mice are also subgrouped based on antibody treatment. Mice are randomly assigned to treatment groups with even distribution by age. Animals injected with anti-iC3b antibodies (2H8, 2A10, 6G1, 5D2 and a control antibody) receive one time per week i.p. injections (3 mg/kg body weight/injection) of the antibody.

Visual function is monitored by analysis of b-wave electroretinograms (ERGs), a reliable measure of retinal activity and visual function (Niemeyer, Digit J Ophthalmol 4(10), 1998). Histological evaluation of sections of whole eyes through the optic nerve head is conducted to reveal pathologic changes in the retinal pigmented epithelium (RPE) and the presence of sub-RPE deposits in APOE4-HFC mice. RPE lesions are exemplified by vacuolization, pyknosis, hyper- and hypo-pigmentation, and infiltrating microglia. RPE damage in APOE4-HFC mice is quantified by immunostaining RPE flat mounts with an antibody to the tight junction protein, zona occludens 1 (ZO-1), staining nuclei with Hoechst 33342, and analyzing the images for RPE size, integrity, and number (Ding et al., Proc Natl Acad Sci USA 108:E279-E287, 2011).

ERG:

ERGs are recorded using the Espion E2 system (Diagnosys LLC) (Ding et al., Vision Res 48:339-345, 2008; Malek, et al., Adv Exp Med Biol 613:165-170, 2008). Data analysis and fitting were performed as described (Henmann et al., J Neurosci 30:3239-3253, 2010). Personnel responsible for ERGs and assessment of pathology are masked to the identity of treatment groups.

Immunohistochemistry:

Mouse posterior eyecups are embedded in agar and vibratome-sectioned at 50-100 μm. Sections are blocked in 10% normal donkey serum (Jackson Immunoresearch), incubated overnight with primary antibodies, incubated for 2 h in Alexa fluorophore-conjugated secondary antibody (Invitrogen), and counterstained with Hoechst 33342 (Invitrogen). Confocal images are acquired using a Leica SP5 laser-scanning confocal microscope.

Histology:

Mice are deeply anesthetized and perfused transcardially with saline followed by fixative (4% paraformaldehyde in phosphate buffer, pH 7.4, for immunohistochemistry or a mixture of 2% paraformaldehyde and 2% glutaraldehyde in phosphate buffer for semithin and ultra-thin sections). For semithin sections, eyeballs are enucleated; the cornea and lenses are removed, dehydrated, embedded in Epon-Spurr resin, cut at 500 nm, mounted on glass slides, and stained with toluidine blue. Sections are examined under a Zeiss Axioplan 2 microscope (Thornwood).

### Example 12

### Staining Human Retinal Pigmented Epithelial (RPE) Cells

RPE Cell Culture:

RPE cells are derived from human fetal eyes (Advanced Bioscience Resources) as described by Hu and Bok (Mol Vis 7:14-19, 2001). Early passage (p1-p2) cells are seeded onto laminin-coated porous supports (Millipore Millicell-HA Culture Plate Inserts, PIHA 01250) and cultured in “Miller medium” (Maminishkis et al., Invest Ophthalmol Vis Sci 47:3612-3624, 2006). One- to 3-mo postseeding, cultures are exposed to complement-competent human sera, human sera depleted of C1q, C5, or Factor B, or human serum depleted of IgG by absorption with protein A/G coupled to agarose beads (Pierce) at 10% (vol/vol) in serum-free Miller medium for 24 h. The specimens are then rinsed in PBS (3×, 15 min), fixed for 10 min in 4% paraformaldehyde in PBS, and stored in 0.4% paraformaldehyde pending sectioning and immunohistochemical analyses.

Immunohistochemistry:

Confocal laser scanning immunofluorescence imaging is performed as described in Anderson et al., Invest Ophthalmol Vis Sci 40:3305-3315, 1999, on 100-μm vibratome sections or flat mounts of fixed RPE cell cultures. Flat mounts are bleached (Melanin Bleach Kit; Polysciences) for 10 min to allow visualization of sub-RPE deposits that would otherwise be obscured by RPE pigment.

Electron Microscopy:

RPE cultures are fixed in 2% glutaraldehyde and 2% formaldehyde, postfixed in 2% osmium tetroxide, dehydrated in a graded ethanol series up to 100% and embedded in LX-112 resin (Ladd Research). Thin sections are stained with 2% uranyl acetate and lead citrate and imaged on a JEOL JEM-1230 transmission electron microscope.

1. A chimeric, humanized, veneered or isolated human antibody that preferentially binds to iC3b relative to C3b and thereby binds to drusen.

2-3. (canceled)

4. The antibody of claim 1 that binds to an epitope of iC3b within SEQ ID NO:2.

5. The antibody of claim 1 that binds to an epitope of iC3b within SEQ ID NO:3.

6. The antibody of claim 1 that binds to a conformational epitope present in iC3b.

7. The antibody of claim 1, wherein the antibody binds to amyloid plaques in brain tissue of a subject with Alzheimer's disease.

8-9. (canceled)

10. The antibody of claim 1 that is an Fab fragment, single chain Fv, or single domain antibody.

11. The antibody of claim 1 wherein the isotype is human IgG1.

12. The antibody of claim 1 having at least one mutation in the constant region.

13. The antibody of claim 12, wherein the mutation reduces complement fixation or activation by the constant region.

14. The antibody of claim 13 having a mutation at one or more of positions 241, 264, 265, 270, 296, 297, 322, 329 and 331 by EU numbering.

15. The antibody of claim 14 having alanine at positions 318, 320 and 322.

16. The antibody of any of claim 1, wherein the isotype is of human IgG2 or IgG4 isotype.

17. The antibody of claim 1 wherein the antibody is cross-reactive with a non-human iC3b.

18. The antibody of claim 17, wherein the antibody is cross-reactive to a murine iC3b.

19. The antibody of claim 1, wherein the antibody is a humanized version of a murine antibody, wherein the murine antibody has a K_{D} at least 10-fold lower for iC3b relative to C3 as determined by surface plasmon resonance.

20. The antibody of claim 1, wherein the antibody is a humanized version of a murine antibody, wherein the murine antibody has a K_{D} at least 2-fold lower for iC3b relative to C3b or C3 as determined by a sandwich ELISA.

21. The antibody of claim 1, wherein the antibody is a humanized version of a murine antibody, wherein the murine antibody has at least five-fold greater affinity for iC3b relative to C3 as determined by immunoprecipitation.

22. The antibody of claim 1, wherein the antibody is a humanized version of a murine antibody, wherein the murine antibody has at least five-fold greater affinity for iC3b relative to C3b as determined by immunoprecipitation.

23. (canceled)

24. A method of treating or effecting prophylaxis of a disease characterized by abnormal levels or distribution of iC3b relative to healthy individuals comprising administering an effective regime of an antibody that preferentially binds to iC3b relative to C3b or an agent that induces such an antibody to a patient having or at risk of a disease associated with iC3b aggregation and thereby treating or effecting prophylaxis of the disease.

25-37. (canceled)

38. A method of inhibiting formation of drusen comprising administering an effective regime of an antibody that preferentially binds to iC3b relative to C3b or an agent that induces such an antibody to a patient having or at risk of a disease associated with drusen formation and thereby inhibiting drusen formation in the patient.

39. A method of inhibiting aggregation of iC3b comprising administering an effective regime of an antibody that preferentially binds to iC3b relative to C3b or an agent that induces such an antibody to a patient having or at risk of a disease associated with iC3b aggregation and thereby inhibiting iC3b aggregation in the patient.

40. A method of stabilizing a non-toxic conformation of iC3b comprising administering an effective regime of an antibody that preferentially binds to iC3b relative to C3b or an agent that induces such an antibody to a patient having or at risk of a disease associated with iC3b and thereby stabilizing a nontoxic conformation of iC3b.

41. A method of clearing drusen comprising administering an effective regime of an antibody that preferentially binds to iC3b relative to C3b or an agent that induces such an antibody to a patient having drusen and thereby clearing drusen from the patient.

42. A method of clearing iC3b comprising administering an effective regime of an antibody that preferentially binds to iC3b relative to C3b or an agent that induces such an antibody to a patient having an abnormally high level of iC3b and thereby clearing iC3b from the patient.

43-47. (canceled)

48. A method of treating or effecting prophylaxis of age related macular degeneration comprising administering an effective regime of an antibody that preferentially binds to iC3b relative to C3 or an agent that induces such an antibody to a patient having or at risk of the disease and thereby treating or effecting prophylaxis of the disease.

49. A method of treating or effecting prophylaxis of Alzheimer's disease comprising administering an effective regime of an antibody that preferentially binds to iC3b relative to C3b or an agent that induces such an antibody, to a patient having or at risk of the disease and thereby treating or effecting prophylaxis of the disease; wherein the antibody stains plaques in immunohistochemical analysis of AD brain.

50-63. (canceled)

64. A method of screening an agent for activity against a disease associated with iC3b, comprising administering the agent to a non-human animal disposed to develop deposits of iC3b, and determining whether the agent inhibits, reduces or delays deposits of iC3b or a consequential sign or symptom of a disease associated with such deposits, wherein the agent is
(i) an antibody that preferentially binds to iC3b relative to C3b; or
(ii) an agent that induces such an antibody.

65-66. (canceled)

67. A method of determining a level of drusen deposits in a patient, comprising administering an antibody that preferentially binds to iC3b relative C3b; and detecting presence of bound antibody in the patient.

68. (canceled)
